(19) United States
(12) Patent Application Publication
(10) Pub. No.: US 2026/0273163 A1
(43) Pub. Date: Sep. 17, 2026

(54) SYSTEM AND METHOD FOR TREATING A GASTROINTESTINAL TRACT OF A SUBJECT

(71) Applicant: THERADRAIN LTD., RAANANA (IL)

(72) Inventors: Michael BORTZ, Kfar Saba (IL); Adam SAGIV, Bnai Atarot (IL); Oded MEIRI, Ram On (IL)

(21) Appl. No.: 19/480,053

(22) PCT Filed: May 30, 2024
(86) PCT No.: PCT/IB2024/055286

§ 371 Date: Oct. 30, 2025

Related U.S. Application Data
(60) Provisional application No. 63/469,560, filed on May 30, 2023.

(51) Int. Cl. A61M 1/00; A61M 25/00; A61M 25/10

(52) U.S. Cl. CPC A61M 1/90; A61M 1/87; A61M 25/007; A61M 25/1002; A61M 25/1011; A61M 25/1018; A61M 2210/105

(57) ABSTRACT

A medical system for applying negative pressure within a gastrointestinal tract of a subject includes an annular longitudinal shaft extending along a longitudinal axis and defining an inner diameter. The inner diameter is sized and configured for accommodating a delivery tool during delivery of the medical system into the body. The shaft includes vacuum-orifices positioned therealong. A plurality of inflatable portions extend outwardly from the shaft. Each inflatable portion has a delivery state and an inflated state. A first channel is in fluid communication with an inner volume of each inflatable portion for delivery of an inflating fluid to the inflatable portion for inflation thereof. A second channel is in fluid communication with the vacuum-orifices for delivery of negative pressure to an environment exterior to the shaft via the vacuum-orifices. At least some of the vacuum-orifices are disposed between two of the inflatable portions.

### BACKGROUND

Wounds in the gastrointestinal tract such as perforations and post-surgical leaks, and particularly in the esophagus, are common in endoscopic and open surgical procedures. The endoluminal location of these wounds and natural wet environment surrounding the wounds make these wounds particularly difficult to treat. Limited treatment options exist for these wounds which have significant morbidity and mortality rates and involve extensive hospital stay.

Vacuum assisted closure (VAC) therapy can increase the rate of wound closure. Negative pressure wound therapy (NPWT) or VAC therapy is the application of sub-atmospheric pressure to acute or chronic wounds to promote the healing of a wound. In theory, creating a negative-pressure in the local wound environment, draws away bacteria, exudate, fluid, and debris tissue from the wound site, increases the rate of healing by promoting blood flow and facilitates localized cell migration and proliferation.

There is a need for improved techniques and devices for assisting in healing of wounds in the GI tract, and particularly in the esophagus, by applying negative pressure to the vicinity of the wounds.

### SUMMARY

Various applications herein relate to medical systems and methods for removal of liquid from a target area in the GI tract, for example to assist in healing of an endoluminal wound in the target area.

In accordance with an aspect of the disclosed technology, there is provided a medical system for applying negative pressure within a gastrointestinal tract of a subject, the medical system including:
(a) an annular longitudinal shaft extending along a longitudinal axis and defining an inner diameter, the inner diameter sized and configured for accommodating a delivery tool therein during delivery of the medical system into the body of the subject, the shaft including a plurality of vacuum-orifices positioned along the shaft;
(b) a plurality of inflatable portions extending outwardly from the shaft, each of the plurality of inflatable portions having a delivery operative state and an inflated operative state when inflated by an inflating fluid;
(c) a first channel in fluid communication with an inner volume of each of the inflatable portions and configured for delivery of the inflating fluid to the inflatable portions for inflation thereof; and
(d) a second channel in fluid communication with the plurality of vacuum-orifices and configured for delivery of negative pressure to an environment exterior to the shaft via the plurality of vacuum-orifices,
   wherein, at least some of the plurality of vacuum-orifices are disposed between at least two of the plurality of inflatable portions.

In accordance with another aspect of the disclosed technology, there is provided a method of applying negative pressure to a portion of the gastrointestinal tract of a subject, the method including:
(a) placing the shaft of the medical device as disclosed herein onto an exterior of a delivery tool;
(b) delivering the delivery tool with the medical device into the gastrointestinal tract of the subject;
(c) removing the delivery tool from the gastrointestinal tract of the subject, while retaining the medical device within the gastrointestinal tract;
(d) inflating the inflatable portions by delivering an inflating fluid thereinto, via the first channel;
(e) coupling the second channel to a source of negative pressure; and
(f) while the medical device is within the gastrointestinal tract, applying negative pressure to the gastrointestinal tract, via the second channel and the plurality of vacuum orifices.

In accordance with an aspect of the disclosed technology, there is provided a medical system for applying negative pressure within a gastrointestinal tract of a subject, the medical system including:
(a) a sleeve extending along a longitudinal axis and defining an inner diameter, the inner diameter sized and configured for accommodating a delivery tool therethrough during delivery of the medical system into the body of the subject, the sleeve including:
   (i) a plurality of sleeve orifices positioned longitudinally along the sleeve surface; and
   (ii) a plurality of inflatable portions positioned along the sleeve, each of the plurality of inflatable portions having:
      a delivery operative state; and
      an inflated operative state when inflated by an inflating fluid,
   wherein, at least some of the plurality of sleeve orifices are positioned between at least two of the plurality of inflatable portions;
(b) a first channel in fluid communication with each of the inflatable portions and configured for delivery of the inflating fluid to the inflatable portions for inflation thereof; and
(c) a second channel in fluid communication with the plurality of sleeve orifices, for delivery of negative pressure to an environment exterior to the sleeve via the plurality of sleeve orifices.

### BRIEF DESCRIPTION OF THE FIGURES

The foregoing discussion will be understood more readily from the following detailed description when taken in conjunction with the accompanying Figures, in which:

FIGS. 1A and 1B are top and side exploded view planar illustrations of a medical system according to an embodiment of the disclosed technology;

FIGS. 2A, 2B, 2C, and 2D are, respectively, a top view perspective illustration, a side view perspective illustration, a top view planar illustration, and a cross-sectional illustration of a shaft forming part of a medical system according to an embodiment of the disclosed technology;

FIGS. 3A, 3B, and 3C, are perspective illustrations of another shaft forming part of a medical system according to another embodiment of the disclosed technology;

FIGS. 4A, 4B, 4C, and 4D are, respectively, a perspective illustration, a side view planar illustration, and two cross-sectionals illustration of various types of sleeves forming part of a medical system according to embodiments of the disclosed technology;

FIGS. 5A, 5B, 5C, 5D, and 5E are, respectively, side, top, and bottom view planar illustrations, and two cross-sectional illustrations of a fluid-delivery adaptor forming part of a medical system according to an embodiment of the disclosed technology;

FIGS. 6A, 6B, and 6C are, respectively, a perspective view illustration, and top and side view planar illustrations of an embodiment of a cover forming part of a medical system according to an embodiment of the disclosed technology;

FIGS. 7A, 7B, and 7C are, respectively, a perspective view illustration, and bottom and side view planar illustrations of another cover forming part of a medical system according to another embodiment of the disclosed technology;

FIGS. 8A and 8B are, respectively, an exploded view illustration and a cross-sectional illustration of an assembly including the shaft, fluid-delivery adaptor, and cover of the medical system according to embodiments of the disclosed technology;

FIGS. 9A and 9B are, respectively, a top perspective view illustration and a sectional illustration of a medical system according to an embodiment of the disclosed technology;

FIGS. 10A and 10B are, respectively, a perspective view illustration and a sectional illustration of a medical system according to another embodiment of the disclosed technology;

FIGS. 11A, 11B, 11C, and 11D are, respectively, a perspective view illustration, a top view planar illustration, a sectional illustration, and a partially cut-away illustration of a medical system according to yet another embodiment of the disclosed technology;

FIG. 11E is a perspective view illustration of a variant of the medical system of FIGS. 11A to 11D, according to yet another embodiment of the disclosed technology;

FIG. 12 is a side view illustration of a medical system according embodiments of the disclosed technology during operation thereof within a gastrointestinal tract of a subject;

FIG. 13 is a side view illustration of a medical system according embodiments of the disclosed technology during delivery thereof into a gastrointestinal tract of a subject;

FIG. 14 is a side view illustration of a device including a plurality of medical system according embodiments of the disclosed technology, in series;

FIG. 15 is a flow chart of a method of treating the gastrointestinal tract of a subject using a medical system according to embodiments of the disclosed technology;

FIGS. 16A and 16B are schematic illustrations of a procedure of deploying a medical system, similar to that of FIG. 1, into the body of a subject, according to embodiments of the disclosed technology; and

FIGS. 17A, 17B, 17C, 17D, and 17E are schematic illustrations of steps of a procedure for maintaining the medical system of FIG. 1 in the body of the subject via a nasal wire or tube.

### DETAILED DESCRIPTION

The principles of the medical systems and methods may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. The invention is capable of other implementations or of being practiced or carried out in various ways. Furthermore, it is to be understood that the phraseology and terminology employed in the disclosure is for the purpose of description and should not be regarded as limiting.

In the context of the present description and claims, the terms “proximal” and “distal” are defined relative to a direction in which the system is deployed into the body of the subject. As such, an element is said to be “proximal” if it is closer to the point at which the system enters the body of the subject than other elements, and is said to be “distal” if it is further from the point at which the system enters the body of the subject than other elements.

In the context of the present description and claims, the term “wound” relates to any form of damage to the tissue, including, but not limited to, a leak, a perforation, a rupture, a tear, a cut, or a fistula in the tissue, for example in the wall of the GI tract.

In the context of the present description and claims, the term “negative pressure” relates to sub-atmospheric pressure, which may be applied, for example, to remove fluid or debris from a bodily lumen.

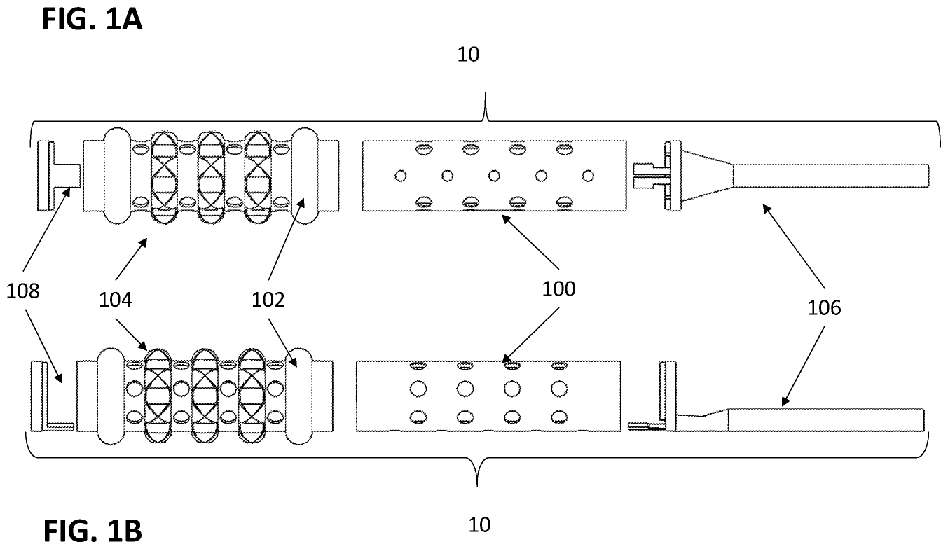

Referring now to the drawings, FIGS. 1A and 1B are, respectively, a top view exploded planar illustration and a side view exploded planar illustration of a medical system 10 according to embodiments of the disclosed technology.

As seen, medical system 10 includes a shaft 100, a plurality of inflatable portions 102, which in some embodiments, and as illustrated, can form part of a sleeve 104, a fluid-delivery adaptor 106, and optionally, a cover 108.

Each of inflatable portion 102 has a delivery operative state (which is typically deflated), and an inflated operative state when inflated by an inflating fluid.

In some embodiments, and as described in further detail hereinbelow, sleeve 104 may be obviated, and inflatable portions 102 may extend from shaft 100 or may be attached (e.g., soldered or adhered) directly onto shaft 100.

In some embodiments, cover 108 may be obviated, for example by providing the functionality of cover 108 as an integral part of shaft 100, by sealing the end of shaft 100.

In some embodiments, the medical system of FIGS. 1A and 1B includes a visualizing component, such as a camera or other visualizing element disposed within the shaft or along a delivery tool used to delivery the system into the body. As such, the system can be visualized throughout deployment thereof, without introduction of additional devices.

In some embodiments, the dimensions of the system, and particularly of the inflatable portions, can be adjusted in vivo and/or in real time, for example in response to changes in the size of a wound being treated by the system. This may be accomplished, for example, by changing a degree of inflation of the inflatable portions.

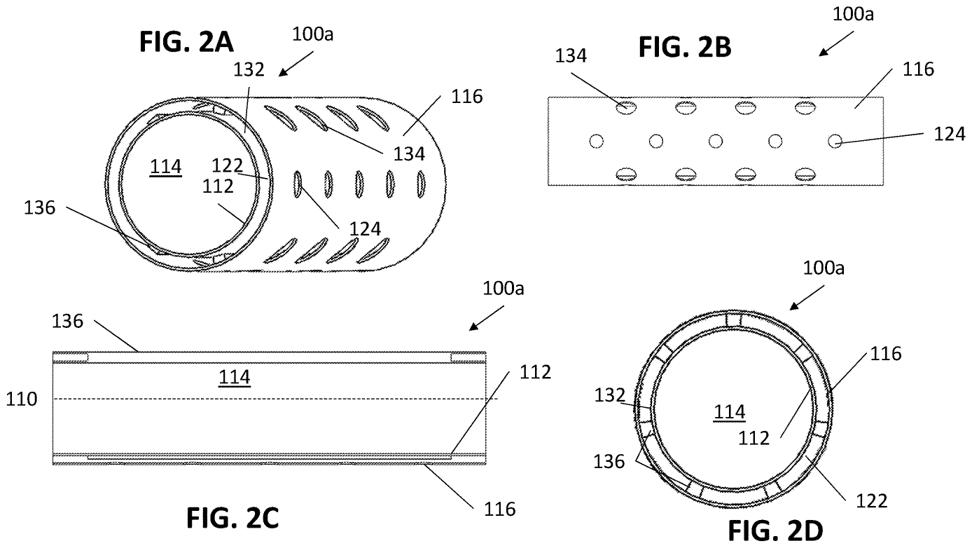

Reference is now made to FIGS. 2A, 2B, 2C, and 2D, which are, respectively, a top view perspective illustration, a side view perspective illustration, a top view planar illustration, and a cross-sectional illustration of a shaft 100a, suitable for forming part of medical system 10 of FIGS. 1A and 1B, according to an embodiment of the disclosed technology.

As seen, shaft 100a is longitudinal and is substantially annular, and is arranged along a longitudinal axis 110. Shaft 100a includes an inner layer, or wall 112 defining a longitudinal hollow 114, and an outer layer, or wall 116. Shaft 100a, or inner layer 112, define an inner diameter Ds, sized and configured for accommodating a delivery tool during delivery of medical system 10 into the body of a subject, as explained in further detail hereinbelow. Delivery tools may include endoscopes such as upper GI endoscopes typically having an outer diameter of more than 7.0 mm or between 8.0 to 12.0 mm. In some embodiments, inner diameter Dₛ is greater than 4.0 mm, greater than 5.0 mm, greater than 6.0 mm, greater than 7.0 mm, or greater than 8.0 mm. In some embodiments, inner diameter Ds is in the range of 4.0 mm to 17.0 mm or 7.0 mm to 15.0 mm. In the some embodiments, an external diameter of shaft 100a is in the range of 6 mm to 25 mm, or in the range of 6 mm to 20 mm.

In some embodiments, and as illustrated, inner layer 112 and outer layer 116 may be circumferential layers, defining a space 118 therebetween.

In some embodiments, and as illustrated, a first longitudinally-extending channel 122, also termed an inflating-fluid delivery channel, is formed between layers 112 and 116, within space 118. In some embodiments, and as illustrated, inflating-fluid delivery channel 122 can be in fluid communication with a plurality of inflation-orifices 124, which are formed in outer layer 116.

In some embodiments, and as illustrated, inflation orifices 124 can be longitudinally distributed along the longitudinal shaft. In some embodiments, inflation orifices 124 are longitudinally distributed in a single line, or forming a single row. In some embodiments, inflation orifices 124 are longitudinally distributed along multiple lines, or more than one row. However, in other embodiments, inflation orifices 124 may be disposed circumferentially about outer layer 116, for example about a single circumference thereof.

In some embodiments, each inflation-orifice 124 has a diameter (in the case of a circular shape) or a longest dimension in the range of 0.5 to 10 mm, 0.1 mm to 2.0 mm, 0.5 mm to 2.0 mm, or 0.5 mm to 1.0 mm.

In some embodiments, and as illustrated, a second longitudinally-extending channel 132, also termed a vacuum-delivery channel, is formed between layers 112 and 116, within space 118. In some embodiments, and as illustrated, multiple such vacuum delivery channels may be formed within shaft 100a.

Vacuum delivery channel(s) 132 is in fluid communication with a plurality of vacuum-orifices 134 which are formed in outer layer 116 and are longitudinally distributed along the longitudinal shaft.

In some embodiments, each orifice 134 has a diameter or longest dimension in the range of 0.5 to 15 mm, 0.5 to 10 mm or 0.5 to 5 mm.

In some embodiments, and as illustrated, inflating-fluid delivery channel 122 can be separated from vacuum-delivery channel(s) 132 by longitudinal supports 136, disposed within space 118 between layers 112 and 116.

In embodiments in which multiple vacuum-delivery channels 132 are formed in shaft 100a, such as the illustrated embodiment, those vacuum delivery channels are also separated by longitudinal supports 136. However, longitudinal supports separating vacuum-delivery channels 132 may enable fluid communication between the vacuum delivery channels, e.g. by including holes or openings within supports 136, or by the supports being slightly shorter than the entire length of the shaft 100a.

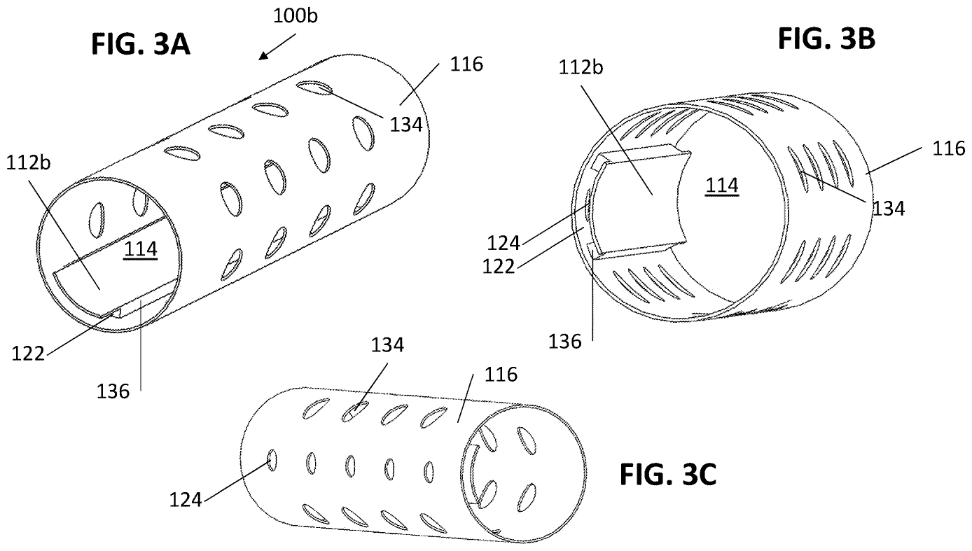

FIGS. 3A, 3B, and 3C are perspective illustrations of another shaft 100b suitable for forming part of medical system 10 of FIGS. 1A and 1B according to another embodiment of the disclosed technology.

Shaft 100b is similar to shaft 100a, with like number representing like elements. Like shaft 100a, also shaft 100b is longitudinal and is substantially annular, and is arranged along longitudinal axis 110.

Shaft 100b includes a circumferential outer layer 116, similar to that of shaft 100a. However, inner layer 112b of shaft 100b only extends in parallel to a portion of outer layer 116. The inner diameter of shaft 100b remains suitable for accommodating a delivery tool, as explained hereinabove.

In shaft 100b, inflating-fluid delivery channel 122 is formed between layers 112b and 116, in a similar manner to that shown with respect to shaft 100a. Inflation-orifices 124 are formed in outer layer 116, in a portion thereof which has inner layer 112b parallel thereto, so that the inflation orifices are in fluid communication with the inflating-fluid delivery channel. Typically, inflation orifices 124 are longitudinally distributed along the longitudinal shaft.

In shaft 100b, hollow 114 is circumscribed, at least in part, by outer layer 116. As such, in addition to being suitable for accommodating a delivery tool, hollow 114 also functions as the vacuum-delivery channel, and is in fluid communication with vacuum-orifices 134.

In some embodiments, and as illustrated, inflating-fluid delivery channel 122 is separated from the vacuum-delivery channel in hollow 114 by longitudinal supports 136, for example disposed at ends of partial inner layer 112b and connecting the inner layer with outer layer 116.

It is to be appreciated that, in some embodiments, the shaft may include additional bores or cavities, not shown in FIGS. 2A to 3C. The additional bores or cavities may be suitable for extension of the inflatable portions therethrough. For example, in some embodiments, in the rest state, the inflatable portions may be disposed within hollow 114 of the shaft, and may be inflated to extend through the additional cavities or bores, so that in the inflated state, the inflatable portions extend outwardly from the shaft.

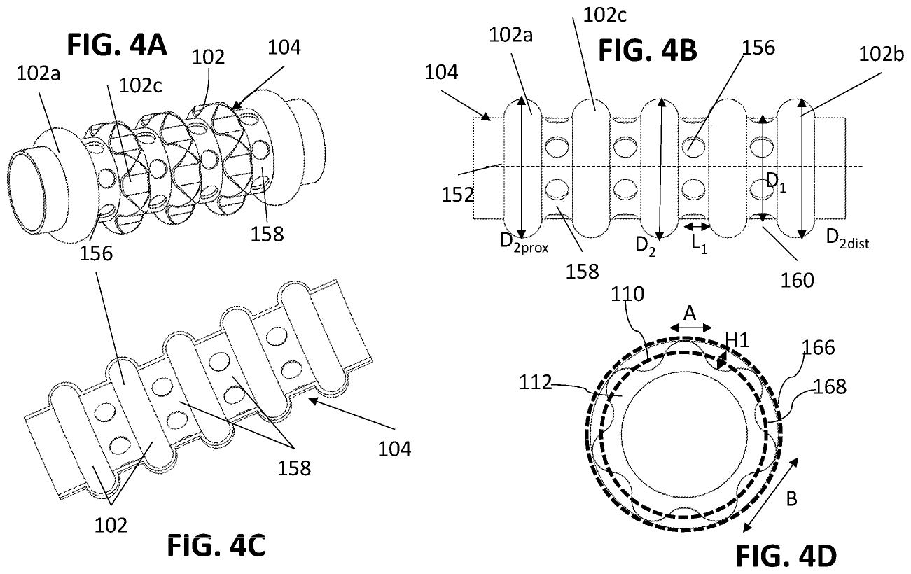

Reference is now additionally made to FIGS. 4A, 4B, 4C, and 4D, which are, respectively, a perspective illustration, a side view planar illustration, and two cross-sectional illustration of various types of sleeves 104 suitable for forming part of medical system 10 according to embodiments of the disclosed technology. The sectional illustration of FIG. 4C is taken along a longitudinal axis of a sleeve 104, which is slightly different form the sleeve shown in FIGS. 4A, 4B, and 4D, as explained hereinbelow. The sectional illustration of FIG. 4D is taken in perpendicular to the longitudinal axis of sleeve 104.

Sleeve 104 is arranged about a longitudinal axis 152. In some embodiments, sleeve 104 has a length L of at least 10 mm, at least 20 mm, or at least 25 mm.

Sleeve 104 includes the plurality of inflatable portions 102, as mentioned hereinabove with respect to FIGS. 1A and 1B. In some embodiments, and as illustrated, inflatable portions 102 can be arranged longitudinally along the surface of sleeve 104.

In some embodiments, sleeve 104 includes at least two inflatable portions 102. However, in other embodiments, sleeve 104 may include three inflatable portions (as shown), or more than three inflatable portions. In some embodiments, sleeve 104 includes at least four inflatable portions 102.

Sleeve 104 further includes a plurality of sleeve orifices 156, which may be positioned longitudinally along the sleeve surface. As explained in further detail hereinbelow, sleeve orifices are configured to deliver negative pressure to an area outside of sleeve 104.

In the illustrated embodiment, inflatable portions 102 are separated by tubular separating segments 158, and sleeve orifices 156 are formed in the separating segments. However, sleeve 104 may have different structures. For example, inflatable portions 102 may form a thread, or screw-shape, around sleeve 104, with sleeve orifices 156 being formed in the recessed portions of the thread. As another example, inflatable portions 102 need not surround the entire circumference of the sleeve, such that the inflatable portions 102 and non-inflatable portions of sleeve 104 may form a ‘checkered’ structure, with the sleeve orifices being disposed in the recessed areas of the ‘checkered’ structure. It is to be appreciated that other shapes of inflatable portions 102, which are adapted to form gaps in which the vacuum orifices are disposed, are considered within the scope of the present application, such that when negative pressure is applied, vacuum pockets are formed within those gaps.

Returning to the illustrated embodiment, each of separating segments 158 has a first diameter DI, in a direction perpendicular to longitudinal axis 152. In some embodiments, first diameter DI is in the range of 6 mm to 25 mm or in the range of 6 mm to 20 mm.

In some embodiments, a length of each separating segment 158, indicated by L1, is in the range of 1 mm to 20 mm or in the range of 2 mm to 20 mm. In some embodiments, the length of the separating element may also be termed the distance between two adjacent inflating portions 102. In some embodiments, LI is not greater than 10 mm, not greater than 8 mm, or not greater than 5 mm.

In some embodiments, each of sleeve orifices 156 has a diameter or longest dimension in the range of 0.5 to 5 mm or 0.5 to 10 mm.

In some embodiments, in the inflated operative state, inflatable portions 102 have a second diameter D2, which is greater than diameter D1 of separating segments 158, such that gaps 160 are formed between the inflatable portions, along longitudinal axis 152.

In some embodiments, an external diameter of inflatable portions 102, in the delivery state, is in the range of 6 mm to 30 mm, or in the range of 6 mm to 25 mm. In some embodiments, the external diameter of inflatable portions 102, in the delivery state, is equal to, or slightly larger (e.g. up to 10% larger) than D1, or than the external diameter of the shaft. In some embodiments, external diameter D2 of inflatable portions 102, in the inflated state, is in the range of 9 mm to 45 mm. In some embodiments, a difference in the external diameter of inflatable portions between the inflated operative state and the delivery operative state is in the range of 3 mm to 35 mm. In some embodiments, D2 depends on the location within the GI tract at which the medical system is deployed. For example, in the upper portion of the GI tract, D2 may be in the range of 9 mm to 35 mm, whereas in the lower portion of the GI tract (e.g., the intestines), D2 may be in the range of 20 mm to 45 mm.

In some embodiments, diameter D2 is equal to all inflatable portions 102.

In other embodiments, diameter D2ₚᵣₒₓ of a proximal-most inflatable portion 102a, and diameter D2_{dist} of a distal-most inflatable portion 102b, in the inflated state, are greater than diameters D2 of other inflatable portions, disposed between inflatable portions 102a and 102b. As explained hereinbelow, in use, such distinction in diameters enables sealing of the sleeve in the area of a target location within the gastrointestinal tract. This sealing provides a controlled length along the gastrointestinal tract in which negative pressure therapy is applied.

In yet other embodiments, diameter D2ₚᵣₒₓ of a proximal-most inflatable portion 102a, and diameter D2_{dist} of a distal-most inflatable portion 102b, in the inflated state, are smaller than diameters D2 of other inflatable portions, disposed between inflatable portions 102a and 102b.

In yet other embodiments, diameter D2ₚᵣₒₓ of a proximal-most inflatable portion 102a, and diameter D2_{dist} of a distal-most inflatable portion 102b, in the inflated state, are substantially equivalent to diameters D2 of other inflatable portions, disposed between inflatable portions 102a and 102b.

In some embodiments, at least some of inflatable portions 102 are ring shaped, and have a circular or annular cross section, as shown for example for inflatable portions 102a and 102b, and as shown in sectional illustration 4C. In some embodiments, the annular inflatable portions may be fully circumferential, i.e., circumscribing the entirety of shaft 100. In some embodiments, the annular inflatable portions may be partially circumferential, i.e., disposed about only part the circumference of shaft 100, such as 50% of the circumference, or most of the circumference.

However, in some other embodiments, inflatable portions 102 may be substantially spherical, for example substantially as described in PCT Patent Application No. PCT/IB2023/062845 filed on Dec. 16, 2023 which is incorporated by reference as if fully set forth herein.

In some embodiments, at least some of inflatable portions 102, for example inflatable portion 102c, include a plurality of longitudinally arranged lobes 161 where each pair of adjacent lobes 161 is separated by a longitudinally arranged trough 162. As such, inflatable portion 102c includes an equal number of lobes 161 and troughs 162.

In some embodiments, inflatable portion 102c includes at least 3, at least 4, at least 5, or at least 6 lobes 161. In some embodiments, lobes 161 are distributed circumferentially about the inflatable portion, typically equidistantly.

In some embodiments, and as illustrated, the lobes 161 of all inflatable portions 102 including such lobes are aligned with one another, and consequently also the troughs 162 are aligned with one another, as seen clearly in FIG. 4A.

In some other embodiments, the inflatable portions 102 may be rotationally offset from one another by half the angular distance between two lobes, which, in the illustrated embodiment, is approximately 30 degrees. In such embodiments, the lobes of a first inflatable portion may be aligned with the troughs of a second, adjacent, inflatable portion. Similarly, the troughs of the first inflatable portion may be aligned with the lobes of the second inflatable portion. In some embodiments, the rotational offset arrangement of the inflatable portions may be an alternating arrangement.

In some embodiments, in the inflated operative state of inflatable portions 102, a height differential between one, or each, of lobes 161, and an adjacent trough 162, indicated by H1 in FIG. 4D, is at least 0.5 mm. In some embodiments, H1 is in the range of 1 to 7 mm, 1 mm to 5 mm, 1 mm to 4 mm, or 1 mm to 3 mm. For clarity, it will be appreciated that the height differential H1 is measured from a point of lobe 161 which is farthest, in a radial direction, from longitudinal axis 152, to a point of trough 162 which is closest, in a radial direction, to longitudinal axis 152. In order to discuss proportions of the lobes 161 and the troughs 162 when an inflatable portion 102 is in the inflated operative state, we imagine a cylinder 166 (seen in FIG. 4D) circumscribing sleeve 104, such that the lobes 161 engage the imaginary cylinder. The imaginary cylinder includes portions that engage inflatable portions 102, indicated by A in FIG. 4D, and portions that are spaced from the inflatable portions, indicated by B.

In some embodiments, a ratio between arcs A and arcs B, in a cross section or end view of inflatable portions 102 (as shown in FIG. 4D), taken at a region of the inflatable portions having a maximal diameter D2 (see FIG. 1B), is at least 5:1, at least 4:1, at least 3:1, at least 2:1, at least 1:1, at least 1:2, at least 1:3, at least 1:4, at least 1:5, at least 1:7, at least 1:10, at least 1:15, at least 1:20, or at least 1:30.

In some embodiments, a portion (A) of the internal wall of imaginary cylinder 166 which engages inflatable portions 102, is less than 60%, 50%, 40%, 30% or 20% of the circumference, or cross-section, of imaginary cylinder 166.

A second imaginary cylinder 168, shown in FIG. 4D, is disposed at half the height H1. The second imaginary cylinder 168 includes arcs, or portions, A½ which cut through lobes 161, and arcs, or portions, B½ which cut through troughs 162. In some embodiments, a ratio between arcs A½ and arcs B½, in a cross section or end view of inflatable portions 168 (as shown in FIG. 4D), is at least 5:1, at least 4:1, at least 3:1, at least 2:1, at least 1:1, at least 1:2, at least 1:3, at least 1:4, at least 1:5, at least 1:7, at least 1:10, at least 1:15, at least 1:20, or at least 1:30.

In some embodiments, at least one of lobes 161 may be textured, or wavy. In some embodiments, lobes 161 may include one or more lobe-troughs, disposed longitudinally along the lobe 161.

In some embodiments, when negative pressure is applied to troughs 162 (as explained herein below), the presence of lobe-troughs enables the negative pressure to be applied from multiple different direction to each location along troughs 162, thus reducing the chance of blockage in troughs 162. Additionally, in use, texturing of lobes 161 may function as a tissue-growth enhancing therapy, for example by mildly massaging a target location at which the sleeve is located.

In some embodiments, sleeve 104 may include a first inflatable portion having a first cross section, e.g. inflatable portion 102a in FIG. 4A, and a second inflatable portion having a second cross section, different from the first cross section, e.g. inflatable portion 104c in the same figure.

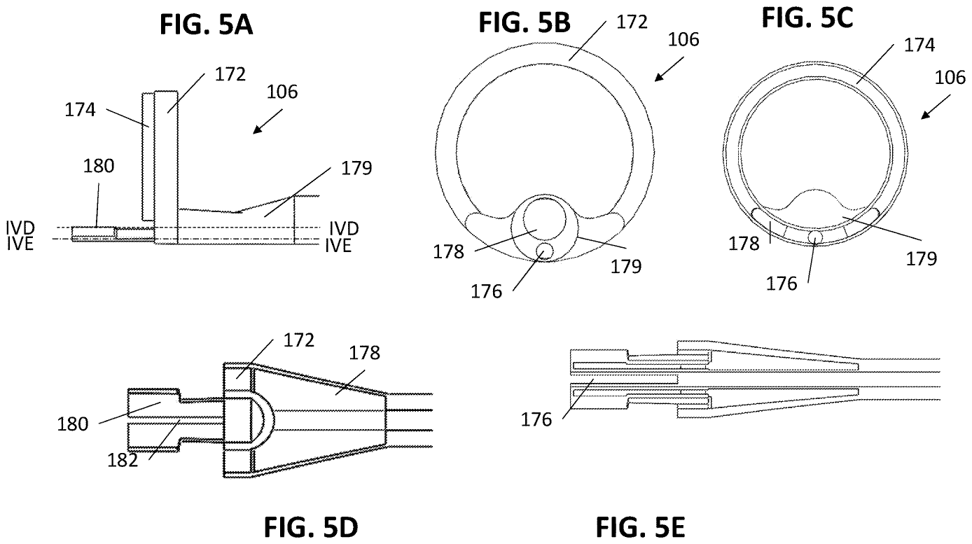

Reference is now made to FIGS. 5A, 5B, 5C, 5D, and 5E, which are, respectively, side, top, and bottom view planar illustrations, and two cross-sectional illustrations of fluid-delivery adaptor 106 suitable for forming part of medical system 10 of FIGS. 1A and 1B according to embodiments of the disclosed technology. The cross-sectional view shown in FIG. 4D is taken along section lines IVD-IVD in FIG. 4A, and the cross-sectional view shown in FIG. 4E is taken along section lines IVE-IVE in FIG. 4A.

Fluid-delivery adaptor 106 includes a generally annular or cylindrical body 172, having wall portion 174 extending distally therefrom. Fluid-delivery adaptor 106 is associated with a first lumen 176, also termed an inflating-fluid delivery lumen and with a second lumen 178, also termed a vacuum delivery lumen.

In the illustrated embodiment, fluid-delivery adaptor 106 is integrally formed with a dual lumen tube 179, defining inflating-fluid delivery lumen 176 and vacuum delivery lumen 178. In other embodiments, the two lumens may be mechanically separate from each other, and may be mechanically separable from fluid-delivery adaptor 106.

In some embodiments, a tab 180 extends distally from cylindrical body 172, at a portion thereof devoid of wall portion 174. Typically, tab 180 extends distally further than wall portion 174. A bore 182 extends from inflating-fluid delivery lumen 176 into, and through, tab 180, such that bore 182 is in fluid communication with inflating fluid delivery lumen 176.

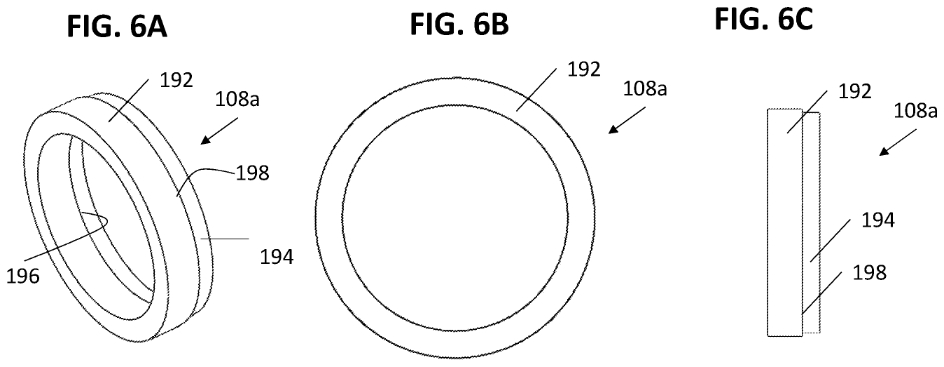

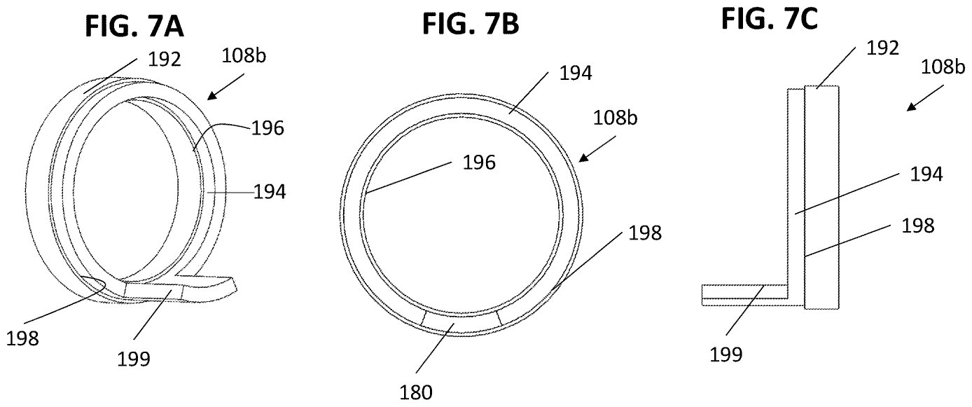

Reference is now made to FIGS. 6A, 6B, and 6C, which are, respectively, a perspective view illustration, and top and side view planar illustrations of an embodiment of a cover 108a suitable for forming part of medical system 10 of FIGS. 1A and 1B, according to an embodiment of the disclosed technology, and to FIGS. 7A, 7B, and 7C, which are, respectively, a perspective view illustration, and bottom and side view planar illustrations of another cover 108b suitable for forming part of medical system 10 according to another embodiment of the disclosed technology.

For brevity, any common components between covers 108a and 108b are described using the same reference numerals, and with respect to cover 108a, generally.

Cover 108a includes a generally annular or cylindrical body 192, having a circumferential wall portion 194 extending proximally therefrom.

In some embodiments, an internal diameter of wall portion 194 is greater than an internal diameter of annular body 192, such that an internal shoulder 196 is formed between the annular body 192 and the wall portion 194. However, in other embodiments, the internal diameter of wall portion 194 may be equal to the internal diameter of annular body 192.

Typically, an external diameter of wall portion 194 is smaller than an external diameter of annular body 192, such that an external shoulder 198 is formed between the annular body 192 and the wall portion 194.

In some embodiments, and as illustrated in FIGS. 7A to 7C with respect to cover 108b, a tab 199 extends proximally from wall portion 194.

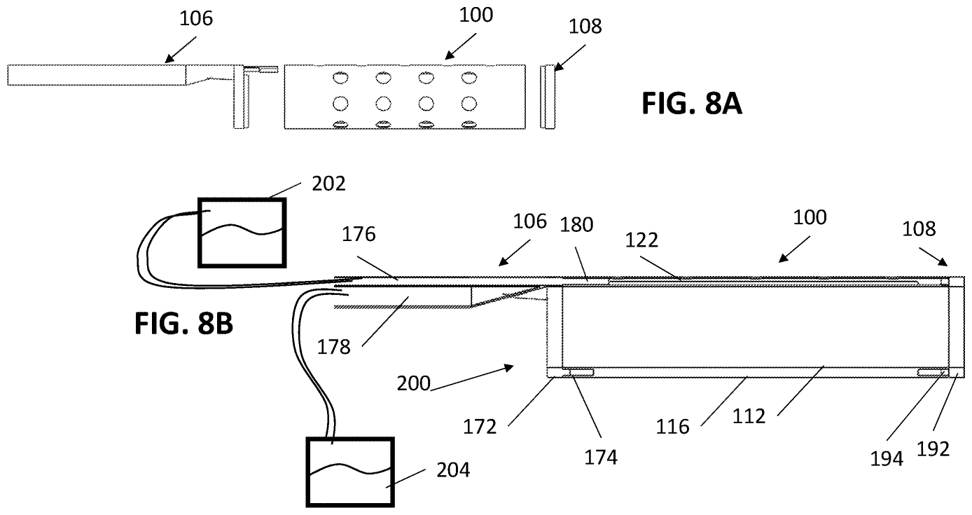

FIGS. 8A and 8B are, respectively, an exploded view illustration and a cross-sectional illustration of an assembly 200 including shaft 100, fluid-delivery adaptor 106, and cover 108 of medical system 10 according to embodiments of the disclosed technology.

As seen, fluid-delivery adaptor 106 is connected to a proximal end of shaft 100, such that wall portion 174 is disposed between inner layer 112 and outer layer 116, and body 172 extends proximally to the shaft. Tab 180 extends into inflating-fluid delivery channel 122 of shaft 100. In some embodiments in which longitudinal supports 136 do not reach the proximal end of shaft 100, such that a gap is formed between wall portion 174 and supports 136. In such embodiments, tab 180 extending into inflating-fluid delivery channel 122 ensures that the inflating fluid delivery channel is fluidly separated from vacuum-delivery channel(s) 132, while the vacuum-delivery channels are in fluid communication with each other.

Similarly, cover 108 is connected to a distal end of shaft 100, such that wall portion 194 is disposed between inner layer 112 and outer layer 116, and body 192 extends distally to the shaft. Additionally, an in a similar manner to that described herein for tab 180 of fluid-delivery adaptor 106, tab 199 of cover may be disposed within inflating-fluid delivery channel 122 of shaft 100, and may assist in separating the inflating-fluid delivery channel 122 from the vacuum delivery channel(s) 132.

However, as mentioned above, in some embodiments cover 108 may be obviated, for example by integrating the functionality thereof into an end of shaft 100. For example, layers 112 and 116 may sealingly engage each other at the distal end of shaft 100, thereby obviating the cover 108.

The connection between fluid-delivery adaptor 106 and shaft 100 is such that first lumen 176 is in fluid communication with inflating-fluid delivery channel 122 and with inflating-orifices 124, and second lumen 178 is in fluid communication with vacuum-delivery channel(s) 132 and with vacuum-orifices 134.

First lumen 176 of fluid-delivery adaptor 106, may be coupled to a source 202 of inflating fluid, such that, in assembly 200, when first lumen 176 is in fluid communication with inflating-fluid delivery channel 122, the inflating-fluid delivery channel is coupled to the source of inflating fluid, and inflating fluid can flow from the source to orifices 124. In some embodiments, the first lumen and the first channel may be decoupled from the source of inflating fluid once the inflatable portions 102 are inflated, while blocking flow of the inflating fluid out of the inflatable portions and/or out of the first channel and/or first lumen.

Similarly, second lumen 178 of fluid-delivery adaptor 106, may be coupled to a source 204 of negative pressure, such that, in assembly 200, when first lumen 178 is in fluid communication with vacuum delivery channel(s) 132, the vacuum fluid delivery channel is coupled to the source of negative pressure, and negative pressure can be applied from the source, via orifices 134, to the environment surrounding the shaft.

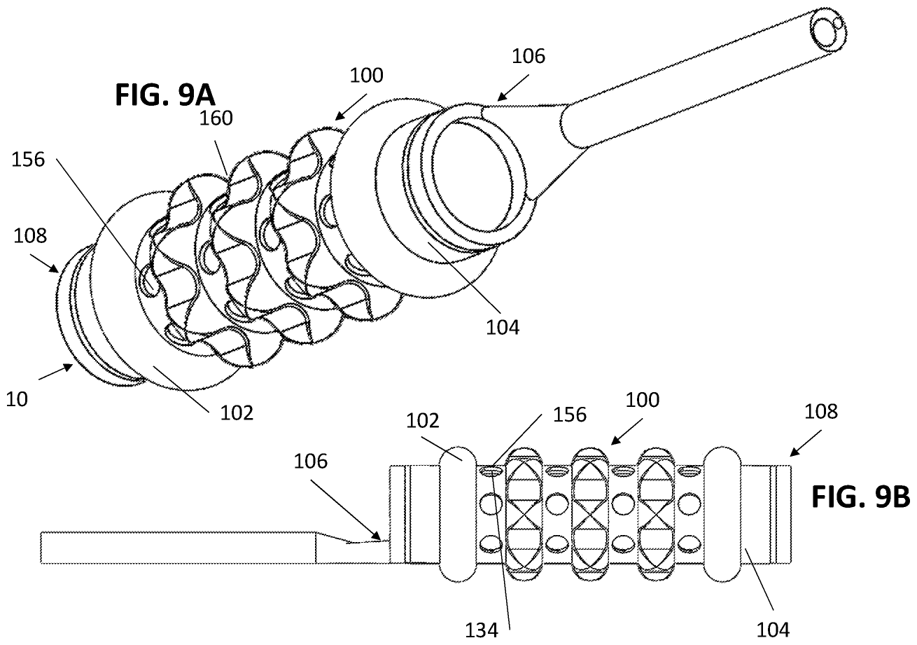

Reference is now made to FIGS. 9A and 9B, which are, respectively, a top perspective view illustration and a side view planar illustration of a medical system 10 according embodiments of the disclosed technology.

As seen, sleeve 104 is disposed about shaft 100, while shaft 100 is within assembly 200 (FIGS. 8A and 8B), such that inflatable portions 102 extend radially outwardly from shaft 100. Sleeve orifices 156 are aligned with the vacuum orifices, such that negative pressure applied through the vacuum-delivery channel(s) is applied to the gaps 160 between inflatable portions 102 and to the environment surrounding system 10.

Additionally, the inflating orifices are longitudinally aligned with inflatable portions 102, such that inflatable portions 102 are in fluid communication with the inflating-fluid delivery channel, via the inflating orifices.

In use, inflatable portions 102 may be transitioned from the delivery state to the inflated state by introduction of inflating fluid from source 202 (FIG. 8A) into the inflatable portions, via inflating-fluid delivery channel 122 and orifices 124.

Additionally, negative pressure is applied from the source 204 of negative pressure (FIG. 8A) to gaps 160 between inflatable portions 102, via vacuum delivery channel 132, vacuum-orifices 134, and sleeve orifices 156. As such, the vacuum is applied to the gaps in a radial direction relative to longitudinal axis 152 of sleeve 104 (FIG. 4B). In some embodiments, the source of negative pressure 204 is configure to apply a negative pressure in the range of 30 to 250 mmHg. In some embodiments, the inflatable portions 102 retain their shape when negative pressure is applied thereto.

It is a particular feature of the disclosed technology that shaft 100 and the inflatable portions 102 are sealingly bonded to each other, for example by soldering, gluing or welding. In embodiments in which the inflatable portions 102 form part of sleeve 104, the sleeve must be sealingly bonded to the shaft, using similar techniques. This is critical to ensure that negative pressure delivered via shaft 100 does not drain inflatable portions 102, and similarly that the gaps between the inflatable portions are not contaminated with inflating fluid leaking from the inflatable portions. As a result, it is particularly important that the inflatable portions 102 be sealed relative to vacuum delivery channel(s) 132. In some embodiments, the shaft 100 and inflatable portions 102 or sleeve 104 are produced and form a single unit without a soldering step, for example by three-dimensional printing thereof as a single piece.

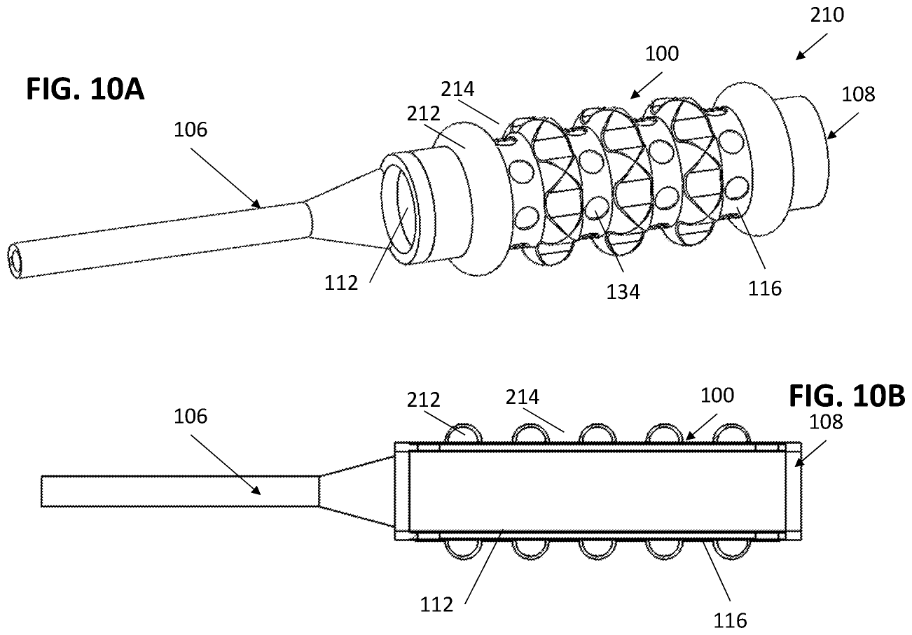

Reference is now made to FIGS. 10A and 10B, which are, respectively, a perspective view illustration and a sectional illustration of a medical system 210 according to another embodiment of the disclosed technology. Medical system 210 is substantially similar to medical system 10, and includes a shaft 100 a fluid-delivery adaptor 106, and a cap 108, substantially as described hereinabove with respect to FIGS. 1A-1B, 2A-3D, 5A-5E, and 6A-7C, respectively.

Medical system 210 differs from medical system 10 in that sleeve 104 is obviated. Instead, each inflatable portion 212 is attached directly to shaft 100, for example by adhering, soldering, or three-dimensional printing of the shaft and the inflatable portions as a single piece. As such, when the inflatable portions are in the inflated state, gaps 214 are formed along shaft 100, between inflatable portions 212. Negative pressure, applied to vacuum-delivery channel 132 is delivered to the environment surrounding the shaft directly from vacuum-orifices 134.

It is to be appreciated that the structural and mechanical characteristics of inflatable portions 212, including materials, material characteristics, dimensions, shapes, and the like, are all equivalent to those described hereinabove with respect to inflatable portions 102 of FIGS. 4A to 4D.

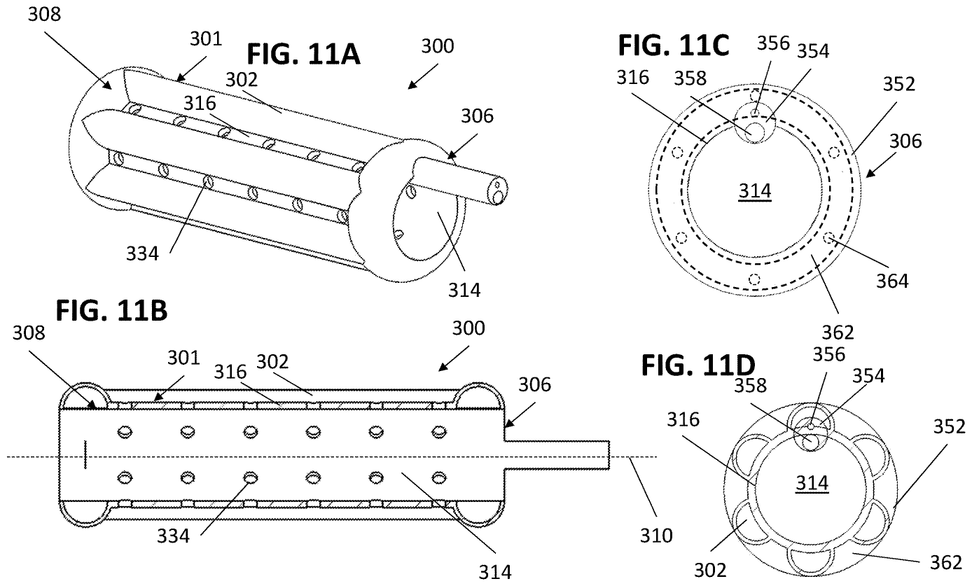

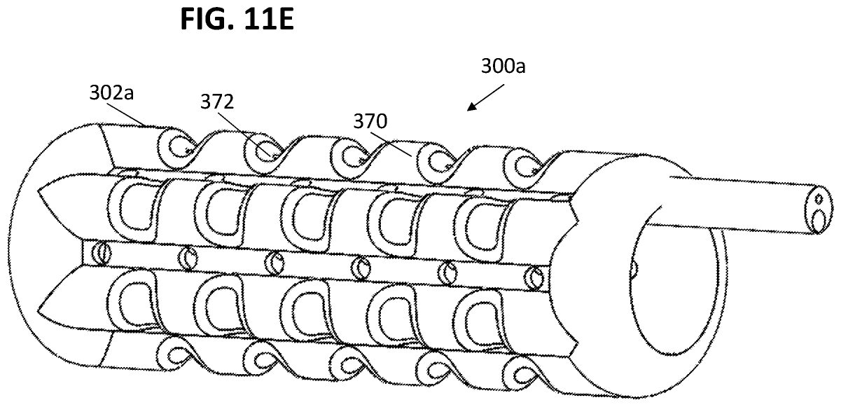

FIGS. 11A, 11B, 11C, and 11D are, respectively, a perspective view illustration, a top view planar illustration, a sectional illustration, and a partially cut-away illustration of a medical system 300 according to yet another embodiment of the disclosed technology. Medical system 300 includes a shaft 301, inflatable portions 302, a fluid-delivery adaptor 306, and a cap 308. Shaft 301 is a longitudinal annular shaft, arranged along a longitudinal axis 310. The shaft includes a single, outer layer 316, which defines a hollow 314 sized and configured for accommodating a working tool therein, in a similar manner to hollow 114 described hereinabove with respect to FIGS. 2A to 3D. In some embodiments, the diameter of hollow 314 is similar to that of the diameter Ds of hollow 114 (FIGS. 2A-2D).

Shaft 301 includes a plurality of vacuum orifices 334, arranged longitudinally along layer 316. Vacuum orifices 334 are similar to vacuum orifices 134 described hereinabove with respect to FIGS. 2A to 3D, and have corresponding dimensions and characteristics. Vacuum orifices 334 are in fluid communication with hollow 314 of shaft 301, such that hollow 314 functions as the vacuum-delivery channel in medical system 300. Negative pressure, applied to hollow 314 is delivered to the environment surrounding the shaft directly from vacuum-orifices 334.

Inflatable portions 302 are longitudinal inflatable portions, each being attached directly to shaft 301 along a longitudinal length thereof, for example by adhering, soldering, or three-dimensional printing of the shaft and the inflatable portions as a single piece. As such, when the inflatable portions are in the inflated state, longitudinal gaps 344 are formed along shaft 301, between inflatable portions 302.

In medical system 300, shaft 301 is devoid of an inflating-fluid delivery channel, and of inflating orifices for delivery of such inflating fluid. As explained herein, the inflating fluid is delivered to inflatable portions 302 directly from fluid-delivery adaptor 306.

Specifically, fluid delivery adaptor 306 includes a generally annular or cylindrical body 352, adapted to engage, or integrally formed with, shaft 301 and inflatable portions 302. Fluid-delivery adaptor 306 further includes a dual lumen tube 354, extending into body 352. The dual lumen tube includes a first lumen 356, also termed an inflating-fluid delivery lumen and with a second lumen 358, also termed a vacuum delivery lumen. Vacuum delivery lumen is in fluid communication with hollow 314 of shaft 301, for delivery of negative pressure thereto.

Inflating-fluid delivery lumen 356 is in fluid communication with an inflating-fluid delivery channel 360, formed within body 352. Inflating fluid delivery channel 362 is in fluid communication with each of inflatable portion 302, for example via an inflation fluid bore 364 formed in body 352, proximally to each inflatable portion 302. In some embodiments, body 352 is hollow, such that a hollow thereof functions as the inflating-fluid delivery channel.

Cover 308 can be hollow and can be in fluid communication with inflatable portions 302, and is adapted for sealing thereof. In embodiments in which the distal end of inflatable portions 302 is sealed, e.g. by direct attachment to shaft 301, cover 308 may be obviated.

In some embodiments, body 352 of fluid-delivery adaptor 306 and/or cover 308 may comprise additional inflatable portions, formed of a similar material to that of inflatable portions 302. In some such embodiments, inflating fluid delivered via inflating-fluid delivery lumen 356 is also used to inflate body 352 and/or cover 308.

In some embodiments, at least part of fluid delivery adaptor 306, inflatable portions 302, and cover 308 may together form a sleeve, adapted to be applied over shaft 301, and then sealed thereto along lateral edges of inflatable portions 302. In such embodiments, body 352 and cover 308 would also be inflatable portions, having similar characteristics to those of inflatable portions 302.

Reference is now made to FIG. 11E, is a perspective view illustration of a medical system 300a, which is a variant of medical system 300 of FIGS. 11A to 11D, according to yet another embodiment of the disclosed technology. Medical system 300a varies from medical system 300 in that inflatable portions 302a thereof includes hills 370 separated by valleys 372. Both hills 370 and valleys 372 are inflated, to the same degree. However, the material thereof is designed so that the diameter of a inflatable portions 302a at valleys 372 is smaller than the diameter of inflatable portions 302a at hills 370.

It is a particular feature of the disclosed technology, and of medical systems 10, 210, and 300, that at least some of the vacuum orifices thereof are each disposed between at least two inflatable portions. In some embodiments, most of the vacuum orifices, or all of the vacuum orifices, are each disposed between at least two inflatable portions.

In is additionally a particular feature of the disclosed technology, and of medical systems 10, 210, and 300, that the distance between each pair of adjacent inflatable portions is not greater than 10 mm, not greater than 8 mm, or not greater than 5 mm.

For the purpose of the present disclosure and claims, the distance between two inflatable portions is defined as the distance between the base of one inflatable portion and a closest point thereto at the base of the other inflatable portion.

For the purpose of the present disclosure and claims, the base of an inflatable portion is the point or area at which, in the inflated state of the inflatable portion, the radial distance between the inflatable portion and the shaft is minimal. When the inflatable portion is connected directly to the shaft, the base of the inflatable portion includes at least the line or area along which the inflatable portion is connected to the shaft.

When the medical system is disposed within the gastrointestinal tract, and negative pressure is applied via the vacuum orifices, the negative pressure naturally draws the tissue of the gastrointestinal tract toward the medical system. The limited distance between adjacent inflatable portions assists in ensuring when the tissue is drawn toward the medical system, the tissue engages the inflatable portions in their inflated state, and is not drawn into the gaps between the inflatable portions. The presence of most, or all, of the vacuum orifices in the gaps between the inflatable portions, in combination with the limited dimensions of those gaps, assists in the formation of vacuum pockets that are not blocked, or occluded, but the tissue being drawn onto the vacuum orifices.

In some embodiments of medical systems 10, 210, and 300, the vacuum delivery channel may be coupled to a source of a fluid, such as a flushing fluid (e.g. air, CO₂, water, or contrast fluids) or a medicament fluid (not explicitly shown). This may occur, for example, when the vacuum delivery channel is not coupled to the source of negative pressure. When the vacuum delivery channel is coupled to the source of fluid, the fluid may be delivered to gaps between inflatable portions 102, in a similar manner to the application of negative pressure thereto. Introduction of such fluids may enable removal of blockages from the vacuum delivery channel, the vacuum orifices, and/or from the sleeve orifices, facilitate viewing of the wound using various imaging technologies, or enhance treatment of the wound.

In some other embodiments, in addition to the vacuum delivery channel, an additional fluid channel may exist (e.g. within the shaft) and may be coupled to the source of the fluid. Additional orifices, e.g. in the shaft, may facilitate fluid communication between the additional fluid channel and an environment surrounding the medical system. In this case, for example, the vacuum delivery channel may be coupled to the source of negative pressure while the additional fluid channel is coupled to a source of the fluid. Introduction of a fluid via the additional fluid channel may enable removal of blockages from the vacuum orifices and/or from the sleeve orifices 106, facilitate viewing of the wound using various imaging technologies, and/or enhance treatment of the wound. In this embodiment, the medical system is configured to continually or periodically deliver fluid and/or negative pressure as needed, without detaching and reattaching the source of negative pressure.

In some embodiments, the fluid may be a flushing fluid. In some embodiments, the fluid may be a medicament fluid, such as an antimicrobial fluid or a tissue-growth promoting fluid. In some embodiments, the fluid may be a contrast fluid. In some embodiments, the fluid may be ionized gas. In some embodiments, the fluid may be carbon dioxide.

In some embodiments, shaft 100 may include an additional longitudinal lumen for delivery of fluid into hollow 114 or to the gastrointestinal tract downstream of shaft 100. For example, such a lumen may be used for delivery of liquid nutrition, typically used in feeding tubes. In some other embodiments, a feeding tube, separate from medical system 10, may be accommodated within, or extend through, hollow 114 of the shaft, for delivery of the liquid nutrition into the hollow or to the gastrointestinal tract downstream of shaft 100.

In some embodiments, the medical system may further include a textured layer (not explicitly shown) such as a layer of mesh, sponge, and the like, enclosing the shaft and inflatable portions.

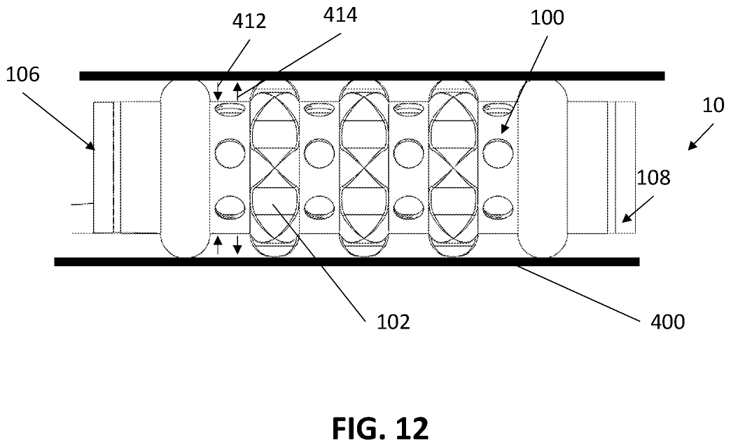

Reference is now additionally made to FIG. 12, which is a side view illustration of medical system 10 during operation thereof within a gastrointestinal tract of a subject. In FIG. 12, system 10 is disposed within a portion 400 of the gastrointestinal tract of a subject, such as within the esophagus or intestine thereof. As seen in FIG. 12, when vacuum is applied to gaps between inflatable portions 102 via vacuum delivery channel 132, orifices 134, and sleeve orifices 156, a force is applied in a first direction, indicated by arrows 412, drawing the walls of esophagus 400 toward system 10.

The structure of inflatable portions 102, and the distances therebetween, ensure that the walls of the esophagus engage the inflatable portions, but do not collapse into gaps between the inflatable portions, thereby ensuring the formation of vacuum pockets in the vicinity of the vacuum orifices (and sleeve orifices). The forces in the direction of arrows 412 may further result in the formation of individual negative-pressure pockets, which maybe improve the suction from the area and reduce the risk of obstruction of orifices by debris.

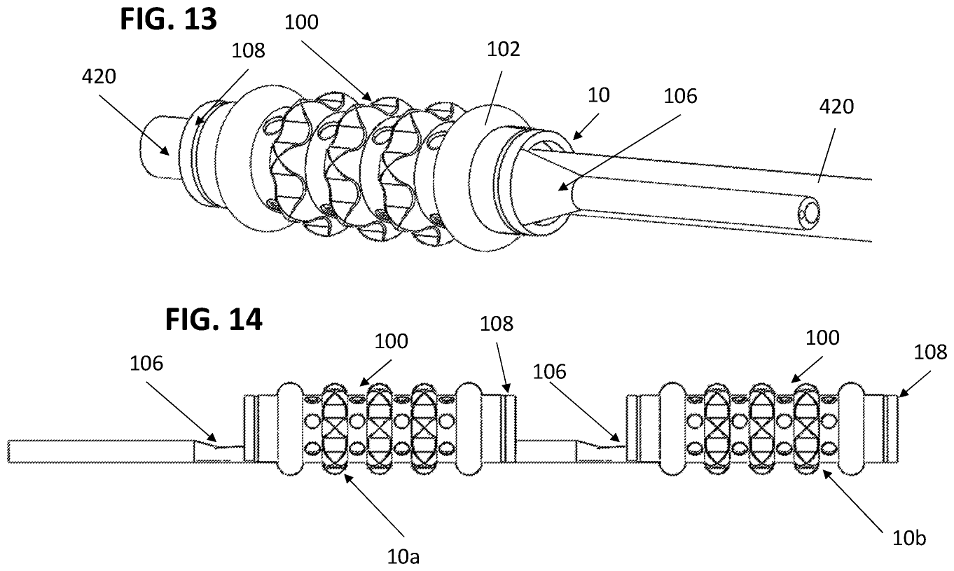

Additionally, because shaft 100 defines hollow 114, at which the pressure is closer to atmospheric pressure, a similar force is applied in a second, opposing direction, as indicated by arrows 414. Forces in the direction of arrow 414 assist in retaining the structure of the medical system and of hollow 114, even when the system, and particularly the shaft, is soft or compliant. Use of a soft or compliant shaft, or medical system, may additionally ease the adaptability of the body to the medical system, and reduce recognition of the medical system as a foreign body. Reference is now made to FIG. 13, which is a side view illustration of medical system 10 according embodiments of the disclosed technology during delivery thereof into a gastrointestinal tract of a subject.

As seen, medical system 10 is delivered into the gastrointestinal tract over a delivery tool 420, such as a scope, e.g. endoscope. The delivery tool extends through hollow 114 of shaft 100. When delivered to the target location in the gastrointestinal tract, delivery tool 420 is detached from system 10 and is extracted from the target location, leaving system 10 placed therein.

For example, during delivery thereof, vacuum acts to bind medical system 10 to delivery tool 420, such that termination of the vacuum facilitates release of the medical system from the delivery tool. As another example, medical system 10 can be mechanically attached to and detached from delivery tool 420, for example by an inflatable portion extending from inner layer 112 of shaft 100 into hollow 114, which inflatable portion is adapted to be inflated to cause the shaft to frictionally engage the delivery tool.

As yet another example, a wire or thread may be incorporated into the shaft. The wire forms a loop, such that one end of the wire is fastened around the delivery tool, and both ends of the wire are held by an operator. When the operator releases one end of the wire and pulls on the other, the shaft, and the entire device, detaches from the delivery tool.

In another example, a wire is threaded through the shaft, with a stopper mechanism disposed at one end of the wire, and the other end of the wire being attached to the vacuum delivery tube. When tension is applied to the wire from one end, the device remains securely in place. When the tension is released, the device is movable toward the distal end of the delivery tool, or endoscope.

In some embodiments, the stopper mechanism can resemble a door stopper, or a plastic snap. In some such embodiments, then the vacuum delivery tube is under tension, the device is immobilized relative to the delivery tool. Upon release of the tension, the device gains freedom of movement toward the distal end of the delivery tool.

In some embodiments, an anchoring mechanism, adapted to anchor the shaft of medical system 10 to the delivery tool, and the releasing mechanism, adapted to release the shaft of medical system 10 from being anchored to the delivery tool, are two separate or distinct mechanism.

In some other embodiments, a single anchoring-and-release mechanism is adapted for anchoring the medical system to the delivery tool and for release of the medical system from the delivery tool when the medical system is suitably placed within the body.

In some embodiments, medical system 10 may be delivered into the gastrointestinal tract of the subject directly, for example via the mouth or via the anus, as explained in further detail hereinbelow.

Reference is now made to FIG. 14, which is a side view illustration of a device 450 including a plurality of medical systems 10 according embodiments of the disclosed technology, in series.

As seen, device 450 includes a pair of medical systems 10a and 10b, as described hereinabove with respect to FIGS. 1 to 9C, where the fluid-delivery adaptor 106b of the distal medical system 10b is attached, in series, to cover 108a of the proximal medical system 10a. It is to be appreciated that additional medical systems 10 may be attached, in a similar manner, to form a device 450 including three or more such medical systems.

It is to be appreciated that, in device 450, only the proximal most medical system, here shown as medical system 10a, is directly coupled to the source of inflating fluid (e.g., a syringe) and the source of negative pressure. Additional medical systems connected in the series, such as medical system 10b, must receive the inflating fluid and negative pressure from the first medical system in the series. As such, in embodiments such as that shown in FIG. 14, the cover 108a (and similarly the cover of any one of the medical systems other than the distal most one) must be modified to enable fluid communication between the inflating-fluid delivery channel 122 of first medical system 10a and the first lumen 176 of the second medical system 10b. Similarly, the cover 108a must be modified to enable fluid communication between the vacuum delivery channel(s) 132 of first medical system 10a and the second lumen 178 of the second medical system 10b.

In some embodiments, medical systems 10a and 10b, and any additional medical systems to be included in device 450, may be connected to one another prior to their insertion into the gastrointestinal tract. In such embodiments, the medical systems may be threaded, in series, onto the delivery tool 420 (see FIG. 13) for delivery thereof.

In other embodiments, medical systems 10a and 10b may each be delivered into the gastrointestinal tract separately, and are connected to each other at the target location.

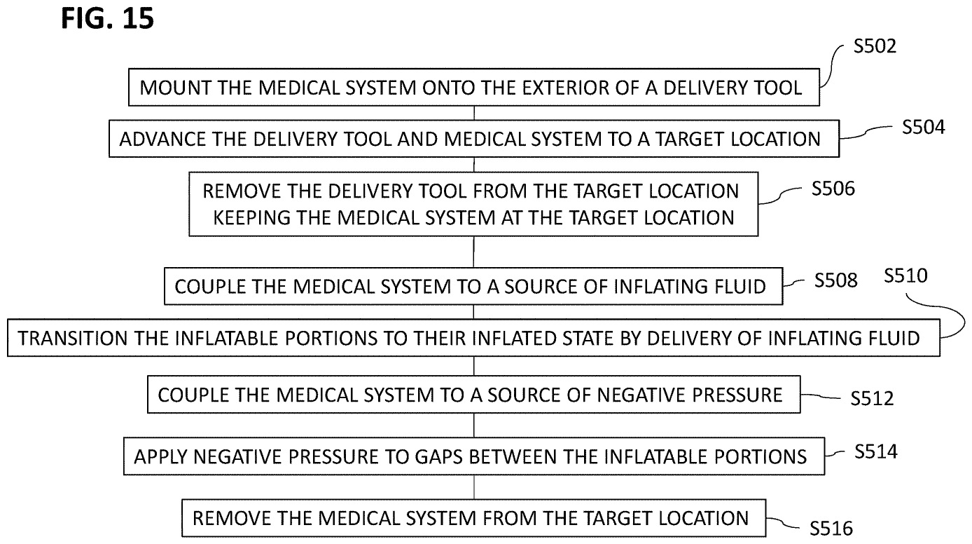

Reference is now made to FIG. 15, which is a flow chart of a method of treating the gastrointestinal tract of a subject using a medical system 10 according to embodiments of the disclosed technology.

At step S502, constructed medical system 10 is mounted onto the exterior of delivery tool 420 at a distal end of the delivery tool, such that the delivery tool advanced, together with medical system 10, to a target location in the gastrointestinal tract of the subject, at step S504. During advancement of the delivery tool, inflatable portions 102 are in the delivery state. For example, the target location may be in the vicinity of a wound in the gastrointestinal tract, which may be an endoluminal or an extraluminal wound. For example, the target location may be in the esophagus, in the intestine, or in the colon, of the subject.

Following delivery of the medical system 10 to the target location, delivery tool 420 (e.g., scope) can be removed from the gastrointestinal tract of the subject, at step S506, while maintaining the medical system 10 at the target location.

At step S508, medical system 10 is coupled to the source 202 of inflating fluid, such as a source of saline, and at step S510 inflatable portions 102 are transitioned from their delivery state to their inflated state, by delivery inflating fluid to the inflatable portions via inflating-fluid delivery channel 122 and inflating orifices 124 of shaft 100. It is to be appreciated that step S508 may occur prior to detachment of the medical device from the delivery tool, or in the illustrated order.

At step S512 source 204 of negative pressure is coupled to medical system 10, and at step S514 the source of negative pressure activated to apply negative pressure to the gaps between inflatable portions 102, via vacuum delivery channel 132, and vacuum orifices 134 (and, in relevant embodiments, sleeve orifices 156). The negative pressure is delivered in a radial direction relative to a longitudinal axis of the medical system.

It is to be appreciated that steps of the method described herein may be carried out in any suitable order. For example, the delivery tool may be removed from the gastrointestinal tract (step S506) immediately following delivery of the medical system to the target location (step S504), after coupling the source of inflating fluid to the medical system (step S508), after inflation of the inflatable portions (step S510), after coupling of the source of negative pressure to the medical system (step S512), or after application of negative pressure (step S514). Similarly, coupling of the medical system to the source of negative pressure (step S512) may occur prior to coupling the source of inflating fluid to the medical system (step S508) or prior to inflation of the inflatable portions (step S510), or may occur following one or both of these steps.

In use, when negative pressure is applied to gaps between inflatable portions 102, the negative pressure is adapted to drain fluid from the target location in the gastrointestinal tract, via vacuum orifices 134. Such draining reduces the amount of contaminating fluids in the target location, and thus can enhance the healing of a wound in the target region.

In some embodiments, application of negative pressure causes the gastrointestinal tract, at the target location, to collapse inward, onto the inflatable portions 102, thereby causing the formation of enclosed negative pressure chambers. Such collapsing also assists in maintaining medical system 10 correctly placed within the target location.

In some embodiments, medical system 10 may be retained at the target location for an extended duration, such as days or weeks.

Following completion of treatment of the target location of the gastrointestinal tract, components of medical system 10, or the entirety thereof, may be removed from the target location at step S516. In some embodiments, such removal further includes deflating of the inflatable portions.

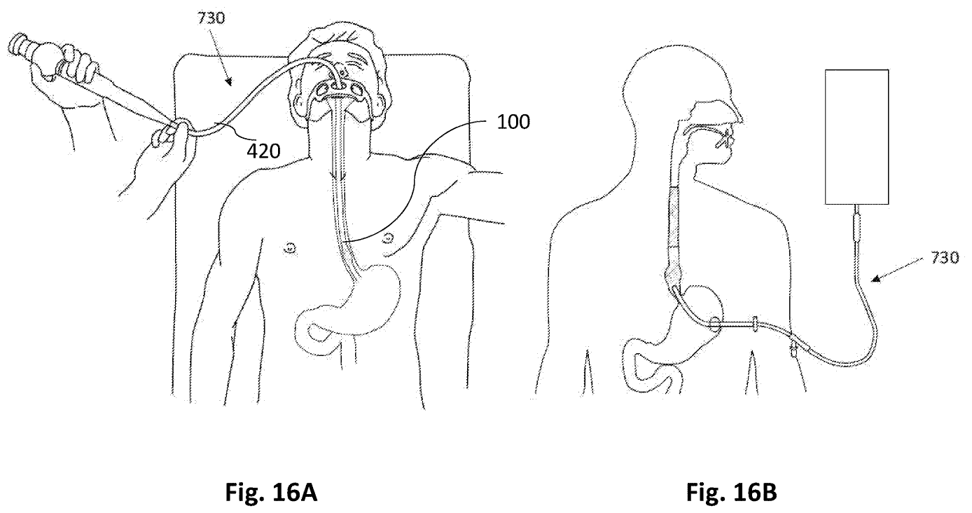

Reference is now made to FIGS. 16A and 16B, which are schematic illustrations of a procedure of deploying portions of a medical system 730, such as any one of medical systems 10, 210, or 300 into the body of a subject, according to embodiments of the disclosed technology.

As seen in FIG. 16A, system 730, and specifically an elongate portion thereof, may be fed into the gastrointestinal tract of the subject, via the subject's mouth. For example, in the illustrated embodiment, an endoscope is used to deliver the elongate tube into the esophagus of the subject, via the subject's mouth.

In FIG. 16B, the elongate portion of medical system 730 is delivered into the esophagus surgically. Specifically, a hole is punctured in the abdominal wall of the subject, and the elongate portion is delivered to the gastrointestinal tract, and specifically to the esophagus, of the subject via the stomach. Alternatively, a hole is punctured in the submental triangle (not shown) of the subject, and the elongate portion is delivered to the gastrointestinal tract and specifically to the esophagus of the subject via the punctured hole. In some such applications, a distal end of elongate the elongate portion may be sharp, or may include a needle, suitable for puncturing the required hole in the abdominal wall. In some other embodiments, the process may be similar to that of placing a percutaneous endoscopic gastronomy (PEG) device, with the distinction that the elongate portion could be delivered further than the stomach, into the esophagus.

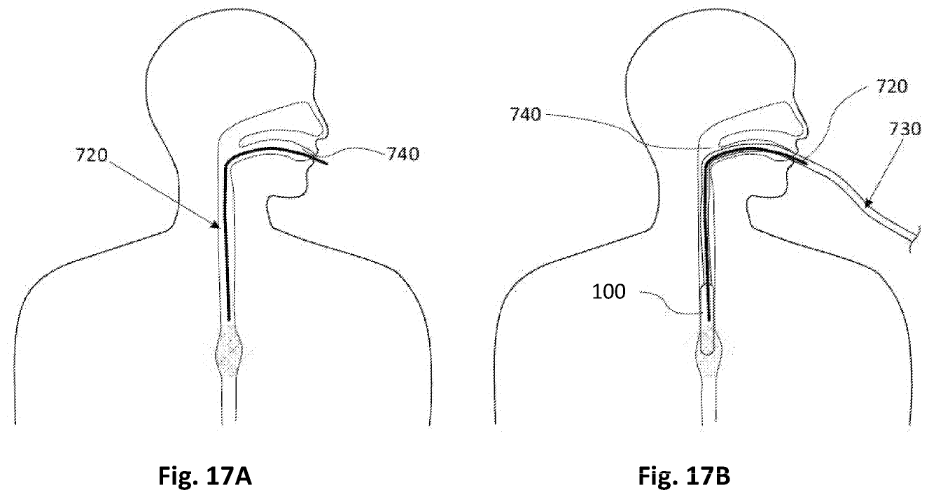

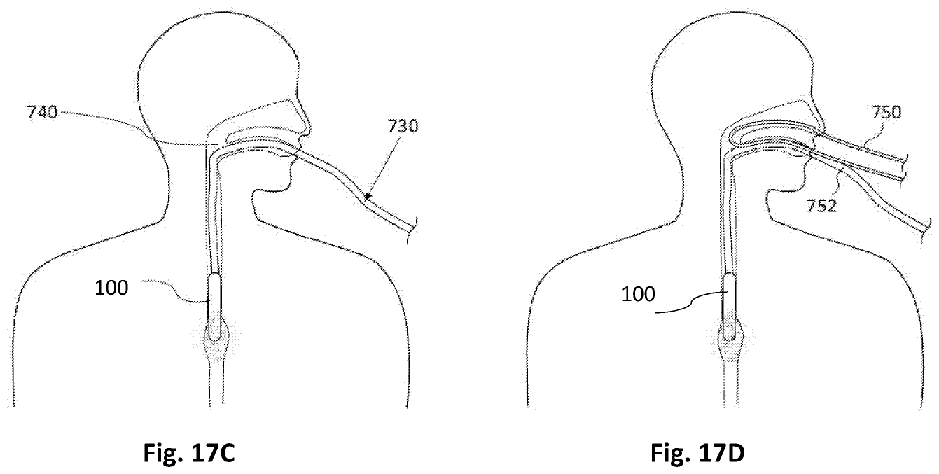

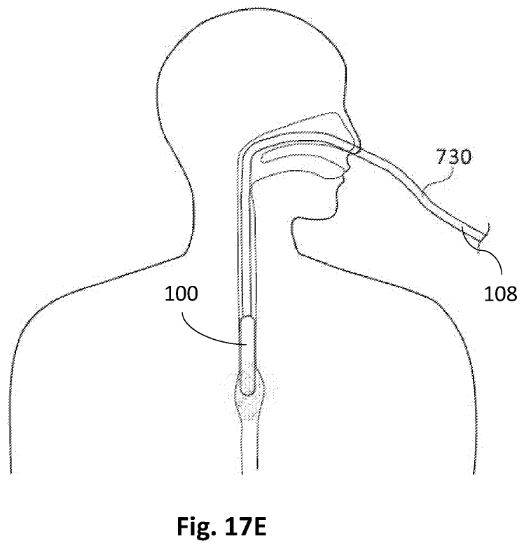

Reference is now made to FIGS. 17A, 17B, 17C, 17D, and 17E, which are schematic illustrations of steps of a procedure for maintaining a portions of medical system 730, such as any one of medical systems 10, 210, or 300 in the body of the subject via a nasal wire or tube, following its introduction as shown in FIG. 16A. For brevity, the elongate portion of medical system 730 is considered to be shaft 100, inflatable portions 102, fluid-delivery adaptor 106, and cap 108 of medical system 10.

As seen, in FIG. 17A delivery device 720, having the elongate portion of medical system 730 disposed thereon, is deployed into the esophagus of the subject, via the subject's mouth 740. In FIG. 17B, the elongate portion of medical system 730 is delivered to the esophagus of the user using the delivery device 720 (not shown). Subsequently, the delivery device 720 is removed from the mouth of the subject, leaving the elongate portion (i.e. shaft 100 and inflatable portions 102) of medical system 730 in place. Following placement of medical system 730 within the esophagus of the subject, delivery device 720 is removed from the body, as seen in FIG. 17C.

In FIG. 17D, a wire 750 is inserted into the nose of the subject, and through the sinuses of the subject into, and out of, the subject's mouth. A proximal end of a tube leading to fluid-delivery adaptor 106, such as a dual lumen tube, is then associated with end 752 of wire 750 extending out of the subject's mouth. In FIG. 17E, the wire 750 is pulled out of the subject's nose. As wire 750 is pulled out of the subject's nose, end 752 of the wire, together with the proximal end of the tube, are pulled into the subject's nose. When wire 750 is fully removed from the subject's nose, the proximal end of the tube continues to extend through the subject's nose and to the exterior of the subject's body, for connection to the source of negative pressure or to a source of inflating fluid, as relevant. The tube connected to the fluid-delivery adaptor 106 can additionally be associated with a nasal retaining element which is configured to maintain the longitudinal position of elongate portion within the subject.

The disclosed technology may be better understood with respect to the following exemplary embodiments:
1. A medical system for applying negative pressure within a gastrointestinal tract of a subject, the medical system including:
(a) an annular longitudinal shaft extending along a longitudinal axis and defining an inner diameter, the inner diameter sized and configured for accommodating a delivery tool having a diameter of at least 2 mm therein during delivery of the medical system into the body of the subject, the shaft including a plurality of vacuum-orifices positioned along the shaft;
(b) a plurality of inflatable portions extending outwardly from the shaft, each of the plurality of inflatable portions having:
   a delivery operative state; and
   an inflated operative state when inflated by an inflating fluid;
(c) a first channel in fluid communication with an inner volume of each of the inflatable portions and configured for delivery of the inflating fluid to the inflatable portions for inflation thereof; and
(d) a second channel in fluid communication with the plurality of vacuum-orifices and configured for delivery of negative pressure to an environment exterior to the shaft via the plurality of vacuum-orifices,
   wherein, at least some of the plurality of vacuum-orifices are disposed between at least two of the plurality of inflatable portions.
2. The medical system of embodiment 1, wherein:
   the annular shaft includes an inner layer and an outer layer;
   the plurality of vacuum-orifices are formed in the outer layer; and
   the second channel is disposed between the inner layer and the outer layer.
3. The medical system of embodiment 2, wherein the inner layer and the outer layer are concentric with each other
4. The medical system of embodiment 1, wherein the shaft further includes a plurality of inflation orifices, each inflation orifice in fluid communication with the first channel and with one of the plurality of inflatable portions, such that fluid communication between the first channel and each of the inflatable portions is via at least one inflation orifice.
5. The medical system of embodiment 4, wherein:
   the annular shaft includes an inner layer and an outer layer, the inner layer and outer layer being concentric with each other;
   the plurality of vacuum orifices and the plurality of inflation orifices are formed in the outer layer; and
   the first channel and the second channel are disposed between the inner layer and the outer layer.
6. The medical system of embodiment 5, wherein the annular shaft includes at least two longitudinal supports, disposed between the inner layer and the outer layer, the supports separating the first channel from the second channel.
7. The medical system of embodiment 4, wherein:
   the annular shaft includes an outer layer and a partial inner layer extending in parallel to a portion of the outer layer;
   the plurality of vacuum orifices are formed in the outer layer;
   the plurality of inflation orifices are formed in a portion of the outer layer which is disposed outwardly of the partial inner layer; and
   the first channel is disposed between the inner layer and the outer layer.
8. The medical system of embodiment 1, wherein the first channel is connected directly to the inner volume of at least one of the inflatable portions for delivery of the inflation fluid thereto.
9. The medical system of any one of embodiments 1, 7, or 8, wherein a central hollow of the annular shaft functions as the second channel.
10. The medical system of any one of embodiments 1 to 9, wherein each of the plurality of inflatable portions includes a partial or complete circumferential inflatable portion, disposed about the shaft.
11. The medical system of any one of embodiments 1 to 9, wherein each of the plurality of inflatable portions includes a longitudinal inflatable portion, extending along at least a portion of the longitudinal axis of the shaft.
12. The medical system of any one of embodiments 1 to 11, wherein each inflatable portion is fixedly attached to an exterior surface of the shaft.
13. The medical system of any one of embodiments 1 to 11, wherein each inflatable portion extends from an interior of the shaft, through a suitable cavity in the wall of the shaft.
14. The medical system of any one of embodiments 1 to 11, wherein each inflatable portion extends from an interior of the inflation channel, outward of the shaft.
15. The medical system of any one of embodiments 4 to 14, further including a sleeve extending along a longitudinal axis and disposed circumferentially about the shaft, the sleeve including:
   (i) a plurality of sleeve orifices positioned longitudinally along the sleeve, and aligned with the plurality of vacuum-orifices, such that the plurality of sleeve orifices are in fluid communication with the second channel via the plurality of vacuum-orifices; and
   (ii) the plurality of inflatable portions, each aligned with one of the inflation orifices of the shaft.
16. The medical system of any one of embodiments 1 to 15, further including an anchoring mechanism, configured for anchoring of the shaft onto the delivery tool during delivery of the medical system into the body of the subject.
17. The medical system of embodiment 16, wherein the anchoring mechanism extends inwardly from the shaft, to engage the delivery tool when the delivery tool is disposed within the shaft.
18. The medical system of any one of embodiments 1 to 17, further including a release mechanism, configured to release the shaft from the delivery tool once the medical system is positioned within the body of the subject.
19. The medical system of embodiment 18, wherein the anchoring mechanism and the release mechanism include a single anchoring-and-release mechanism.
20. The medical system of any one of embodiments 1 to 19, wherein the shaft includes separating segments, separating each pair of adjacent ones of the inflatable portions, and wherein at least some of the vacuum-orifices are disposed along the separating segments.
21. The medical system of any one of embodiments 1 to 20, wherein a maximal distance between each pair of adjacent ones of the plurality of inflatable portions is not greater than 10 mm, not greater than 8 mm, or not greater than 5 mm.
22. The medical system of any one of embodiments 1 to 21, being configured to remain within the gastrointestinal tract for treatment thereof, following detachment from the delivery tool and removal of the delivery tool from the gastrointestinal tract.
23. The medical system of any one of embodiments 1 to 22, further including a fluid-delivery adaptor, adapted to engage a proximal end of each of the first channel and the second channel, the fluid-delivery adaptor being functionally associated with a first lumen and with a second lumen,
wherein the first channel is adapted to couple, via the first lumen, to a source of the inflating fluid, and
wherein the second channel is adapted to couple, via the second lumen, to a source of negative pressure, for delivery of negative pressure to the plurality of vacuum-orifices.
24. The medical system of embodiment 23, wherein the fluid-delivery adaptor is integrally formed with a dual lumen tube including the first lumen and the second lumen.
25. The medical system of embodiment 19 or embodiment 24, wherein the fluid-delivery adaptor includes a separating tab, extending distally into the shaft and adapted to block fluid communication between the first channel and second channel.
26. The medical system of any one of embodiments 1 to 25, further including a cover, adapted to be disposed on a distal end of the first channel and of the second channel for sealing thereof.
27. The medical system of embodiment 26, wherein the cover is adapted for connection to a shaft or a fluid-delivery adaptor of another the medical system, to form a series of the medical systems.
28. The medical system of any one of embodiments 1 to 27, wherein the second channel includes multiple second channels, in fluid communication with each other.
29. The medical system of any one of embodiments 1 to 28, wherein a length of the shaft is at least 20 mm
30. The medical system of any one of embodiments 1 to 29, wherein the plurality of inflatable portions includes at least 3 inflatable portions.
31. The medical system of any one of embodiments 1 to 29, wherein the plurality of inflatable portions includes at least 4 inflatable portions.
32. The medical system of any one of embodiments 1 to 29, wherein the plurality of inflatable portions includes at least 5 inflatable portions.
33. The medical system of any one of embodiments 1 to 32, wherein an inner diameter of the shaft is in the range of 4.0 mm to 17.0 mm.
34. The medical system of any one of embodiments 1 to 32, wherein an inner diameter of the shaft is in the range of 6.0 mm to 15.0 mm.
35. The medical system of any one of embodiments 1 to 32, wherein an inner diameter of the shaft is in the range of 8.0 mm to 12.0 mm.
36. The medical system of any one of embodiments 1 to 32, wherein an inner diameter of the shaft is at least 4.0 mm.
37. The medical system of any one of embodiments 1 to 32, wherein an inner diameter of the shaft is at least 6.0 mm.
38. The medical system of any one of embodiments 1 to 32, wherein an inner diameter of the shaft is at least 8.0 mm.
39. The medical system of any one of embodiments 1 to 38, wherein an external diameter of the shaft is in the range of 6 mm to 25 mm.
40. The medical system of any one of embodiments 1 to 38, wherein an external diameter of the shaft is in the range of 6 mm to 20 mm.
41. The medical system of any one of embodiments 1 to 40, wherein an external diameter of the inflatable portions, in the delivery operative state, is in the range of 6 mm to 30 mm.
42. The medical system of any one of embodiments 1 to 41, wherein an external diameter of the inflatable portions, in the inflated operative state, is in the range of 9 mm to 45 mm.
43. The medical system of any one of embodiments 1 to 42, wherein a difference in the external diameter of inflatable portions, between the inflated operative state and the delivery operative state, is in the range of 3 mm to 35 mm.
44. The medical system of any one of embodiments 1 to 43, wherein the plurality of inflatable portions includes at least three inflatable portions, and wherein, in the inflated operative state, a proximal-most inflatable portion and a distal-most inflatable portion have a greater external diameter than the rest of the plurality of inflatable portions disposed between the proximal most inflatable portion and the distal most inflatable portion.
45. The medical system of any one of embodiments 1 to 44, wherein, in the inflated operative state, a cross section of at least one of the inflatable portions is annular.
46. The medical system of any one of embodiments 1 to 44, wherein, in the inflated operative state, at least one of the inflatable portions is spherical.
47. The medical system of any one of embodiments 1 to 44, wherein, in the inflated operative state, a cross section of at least one of the inflatable portions includes a plurality of lobes, each pair of adjacent lobes separated by a trough.
48. The medical system of any one of embodiments 1 to 44 or 47, wherein, in the inflated operative state, at least one of the inflatable portions includes a plurality of longitudinally arranged lobes and a plurality of longitudinally arranged troughs, wherein each pair of adjacent longitudinally arranged lobes is separated by one of the plurality of longitudinally arranged troughs.
49. The medical system of embodiment 48, wherein, in the inflated operative state of the inflatable body, a height differential between one of the plurality of lobes, and an adjacent one of the plurality of troughs, is in the range of 1 mm to 10 mm.
50. The medical system of embodiment 48, wherein, in the inflated operative state of the inflatable body, a height differential between one of the plurality of lobes, and an adjacent one of the plurality of troughs, is in the range of 2 mm to 10 mm.
51. The medical system of embodiment 48, wherein, in the inflated operative state of the inflatable body, a height differential between one of the plurality of lobes, and an adjacent one of the plurality of troughs, is in the range of 2 mm to 7 mm.
52. The medical system of any one of embodiments 47 to 51, wherein the number of lobes in the plurality of lobes is at least 3.
53. The medical system of any one of embodiments 47 to 51, wherein the number of lobes in the plurality of lobes is at least 4.
54. The medical system of any one of embodiments 47 to 51, wherein the number of lobes in the plurality of lobes is at least 5.
55. The medical system of any one of embodiments 47 to 51, wherein the number of lobes in the plurality of lobes is at least 6.
56. The medical system of any one of embodiments 47 to 55, wherein the lobes are distributed circumferentially about the at least one inflatable portion.
57. The medical system of any one of embodiments 47 to 56, wherein the lobes are equidistantly arranged circumferentially about the at least one inflatable portion.
58. The medical system of any one of embodiments 47 to 57, wherein a radially outward surface of at least one of the lobes is textured or wavy.
59. The medical system of any one of embodiments 1 to 58, wherein, in the inflated operative state, a first inflatable portion of the plurality of inflatable portions has a first cross section in a direction perpendicular to the longitudinal axis of the shaft, and a second inflatable portion of the plurality of inflatable portions has a second cross section in the direction perpendicular to the longitudinal axis of the shaft, the second cross section being distinct from the first cross section.
60. The medical system of any one of embodiments 1 to 59, wherein, introduction of the inflating fluid into the plurality of inflatable portions, via the first channel, transitions each of the inflatable portions from the delivery operative state to the inflated operative state.
61. The medical system of any one of embodiments 1 to 60, wherein the inflating fluid includes pressurized saline.
62. The medical system of any one of embodiments 3 to 61, wherein each of the inflation orifices has a diameter or a longest dimension in the range of 0.5 mm to 10.0 mm.
63. The medical system of any one of embodiments 3 to 61, wherein each of the inflation orifices has a diameter or a longest dimension in the range of 0.5 mm to 5.0 mm.
64. The medical system of any one of embodiments 3 to 61, wherein each of the inflation orifices has a diameter or a longest dimension in the range of 0.5 mm to 2.0 mm.
65. The medical system of any one of embodiments 3 to 61, wherein each of the inflation orifices has a diameter or a longest dimension in the range of 0.5 mm to 1.0 mm.
66. The medical system of any one of embodiments 3 to 61, wherein each of the inflation orifices has a diameter or a longest dimension in the range of 0.1 mm to 2.0 mm.
67. The medical system of any one of embodiments 1 to 65, wherein, when not coupled to a source of negative pressure, the second channel can be coupled to a source of a fluid, for delivery of the fluid via the plurality of vacuum-orifices.
68. The medical system of any one of embodiments 1 to 65, further including a third channel, associated with a third plurality of orifices formed in the shaft, the third channel adapted to be coupled to a source of a fluid, for delivery of the fluid into the gastrointestinal tract via the third plurality of orifices.
69. The medical system of embodiment 67 or embodiment 68, wherein the fluid is a medicament fluid.
70. The medical system of embodiment 67 or embodiment 68, wherein the fluid is a contrast fluid.
71. The medical system of embodiment 67 or embodiment 68, wherein the fluid is ionized gas.
72. The medical system of any one of embodiments 1 to 71, further including a textured layer covering an exterior of the sleeve or of at least some of the inflatable portions.
73. A method of applying negative pressure to a portion of the gastrointestinal tract of a subject, the method including:
(a) placing the shaft of the medical device of any one of embodiments 1 to 72 onto an exterior of a delivery tool;
(b) delivering the delivery tool with the medical device into the gastrointestinal tract of the subject;
(c) removing the delivery tool from the gastrointestinal tract of the subject, while retaining the medical device within the gastrointestinal tract;
(d) inflating the inflatable portions by delivering an inflating fluid thereinto, via the first channel;
(e) coupling the second channel to a source of negative pressure; and
(f) while the medical device is within the gastrointestinal tract, applying negative pressure to the gastrointestinal tract, via the second channel and the plurality of vacuum orifices.
74. The method of embodiment 73, wherein step (c) occurs prior to step (d).
75. The method of embodiment 73, wherein step (c) occurs after step (d).
76. The method of embodiment 73, wherein step (c) occurs prior to step (e).
77. The method of embodiment 73, wherein step (c) occurs after step (e).
78. The method of embodiment 73, wherein step (c) occurs prior to step (f).
79. The method of embodiment 73, wherein step (c) occurs after step (f).
80. The method of embodiment 73, wherein step (d) occurs prior to step (e).
81. The method of embodiment 73, wherein step (d) occurs after step (e).
82. The method of embodiment 73, wherein step (d) occurs prior to step (f).
83. The method of any one of embodiments 73 to 82, wherein:
   the inflating includes inflating at least some of the inflatable portions to engage an internal surface of the gastrointestinal tract and to apply pressure thereto, in a radial direction; and
   the applying negative pressure includes forming multiple negative pressure chambers, between the sleeve and the internal surface of the gastrointestinal tract.
84. A medical system for applying negative pressure within a gastrointestinal tract of a subject, the medical system including:
(a) a sleeve extending along a longitudinal axis and defining an inner diameter, the inner diameter sized and configured for accommodating a delivery tool therethrough during delivery of the medical system into the body of the subject, the sleeve including:
   (i) a plurality of sleeve orifices positioned longitudinally along the sleeve surface; and
   (ii) a plurality of inflatable portions positioned along the sleeve, each of the plurality of inflatable portions having:
      a delivery operative state; and
      an inflated operative state when inflated by an inflating fluid,
   wherein, at least some of the plurality of sleeve orifices are positioned between at least two of the plurality of inflatable portions;
(b) a first channel in fluid communication with each of the inflatable portions and configured for delivery of the inflating fluid to the inflatable portions for inflation thereof; and
(c) a second channel in fluid communication with the plurality of sleeve orifices, for delivery of negative pressure to an environment exterior to the sleeve via the plurality of sleeve orifices.

It should be understood that the use of “and/or” is defined inclusively such that the term “a and/or b” should be read to include the sets: “a and b,” “a or b,” “a,” “b.”

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some applications, sterilizing one or more of any of the various systems, devices, apparatuses, etc. in this disclosure.

Any of the techniques, methods, operations, steps, etc. described or suggested herein can be performed on a living animal (e.g., human, other mammal, etc.) or on a non-living simulation, such as a cadaver, an ex-vivo organ, and/or a simulator device (which may include computerized and/or physical representations of body parts, tissue, etc.).

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver gastrointestinal tract, simulator (e.g., with the body parts, tissue, etc. being simulated), etc.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

1. A medical system for applying negative pressure within a gastrointestinal tract of a subject, the medical system comprising:
(a) an annular longitudinal shaft extending along a longitudinal axis and defining an inner diameter, the inner diameter sized and configured for accommodating a delivery tool having a diameter greater than 2 mm therein during delivery of the medical system into the body of the subject, the shaft including a plurality of vacuum-orifices positioned along the shaft;
(b) a plurality of inflatable portions extending outwardly from the shaft, each of the plurality of inflatable portions having:
a delivery operative state; and
an inflated operative state when inflated by an inflating fluid;
(c) a first channel in fluid communication with an inner volume of each of the inflatable portions and configured for delivery of the inflating fluid to the inflatable portions for inflation thereof; and
(d) a second channel in fluid communication with the plurality of vacuum-orifices and configured for delivery of negative pressure to an environment exterior to the shaft via the plurality of vacuum-orifices,
wherein, at least some of the plurality of vacuum-orifices are disposed between at least two of the plurality of inflatable portions.

2. The medical system of claim 1, wherein:
the annular shaft comprises an inner layer and an outer layer;
the plurality of vacuum-orifices are formed in the outer layer; and
the second channel is disposed between the inner layer and the outer layer.

3. The medical system of claim 1, wherein said shaft further includes a plurality of inflation orifices, each inflation orifice in fluid communication with the first channel and with one of the plurality of inflatable portions, such that fluid communication between the first channel and each of the inflatable portions is via at least one inflation orifice.

4. The medical system of claim 3, wherein:
the annular shaft comprises an inner layer and an outer layer, the inner layer and outer layer being concentric with each other;
the plurality of vacuum orifices and the plurality of inflation orifices are formed in the outer layer; and
the first channel and the second channel are disposed between the inner layer and the outer layer.

5. The medical system of claim 3, wherein:
the annular shaft comprises an outer layer and a partial inner layer extending in parallel to a portion of the outer layer;
the plurality of vacuum orifices are formed in the outer layer;
the plurality of inflation orifices are formed in a portion of the outer layer which is disposed outwardly of the partial inner layer; and
the first channel is disposed between the inner layer and the outer layer.

6. The medical system of any one of claims 1 or 5, wherein a central hollow of the annular shaft functions as the second channel.

7. The medical system of any one of claims 1 to 6, wherein each of the plurality of inflatable portions comprises a partial or complete circumferential inflatable portion, disposed about the shaft.

8. The medical system of any one of claims 1 to 6, wherein each of the plurality of inflatable portions comprises a longitudinal inflatable portion, extending along at least a portion of the longitudinal axis of the shaft.

9. The medical system of any one of claims 1 to 8, wherein each inflatable portion is fixedly attached to an exterior surface of the shaft.

10. The medical system of any one of claims 3 to 9, further comprising a sleeve extending along a longitudinal axis and disposed circumferentially about the shaft, the sleeve comprising:
(i) a plurality of sleeve orifices positioned longitudinally along the sleeve, and aligned with the plurality of vacuum-orifices, such that the plurality of sleeve orifices are in fluid communication with the second channel via the plurality of vacuum-orifices; and
(ii) the plurality of inflatable portions, each aligned with one of the inflation orifices of the shaft.

11. The medical system of any one of claims 1 to 10, further comprising an anchoring mechanism, configured for anchoring of the shaft onto the delivery tool during delivery of the medical system into the body of the subject.

12. The medical system of any one of claims 1 to 11, wherein the shaft includes separating segments, separating each pair of adjacent ones of the inflatable portions, and wherein at least some of the vacuum-orifices are disposed along the separating segments.

13. The medical system of any one of claims 1 to 12, wherein a maximal distance between each pair of adjacent ones of said plurality of inflatable portions is not greater than 10 mm, not greater than 8 mm, or not greater than 5 mm.

14. The medical system of any one of claims 1 to 13, further comprising a fluid-delivery adaptor, adapted to engage a proximal end of each of the first channel and the second channel, the fluid-delivery adaptor being functionally associated with a first lumen and with a second lumen,
wherein the first channel is adapted to couple, via the first lumen, to a source of the inflating fluid, and
wherein the second channel is adapted to couple, via the second lumen, to a source of negative pressure, for delivery of negative pressure to the plurality of vacuum-orifices.

15. The medical system of claim 14, wherein the fluid-delivery adaptor is integrally formed with a dual lumen tube including the first lumen and the second lumen.

16. The medical system of claim 14 or claim 15, wherein the fluid-delivery adaptor includes a separating tab, extending distally into the shaft and adapted to block fluid communication between the first channel and second channel.

17. The medical system of any one of claims 1 to 16, further comprising a cover, adapted to be disposed on a distal end of the first channel and of the second channel for sealing thereof.

18. The medical system of claim 17, wherein the cover is adapted for connection to a shaft or a fluid-delivery adaptor of another said medical system, to form a series of said medical systems.

19. The medical system of any one of claims 1 to 18, wherein a length of the shaft is at least 20 mm.

20. The medical system of any one of claims 1 to 19, wherein the plurality of inflatable portions includes at least 3, at least 4, or at least 5 inflatable portions.

21. The medical system of any one of claims 1 to 20, wherein an inner diameter of the shaft is in the range of 6.0 mm to 15.0 mm.

22. The medical system of any one of claims 1 to 21, wherein an external diameter of the shaft is in the range of 6 mm to 20 mm.

23. The medical system of any one of claims 1 to 22, wherein an external diameter of the inflatable portions, in the delivery operative state, is in the range of 6 mm to 30 mm.

24. The medical system of any one of claims 1 to 23, wherein a difference in the external diameter of inflatable portions, between the inflated operative state and the delivery operative state, is in the range of 3 mm to 35 mm.

25. The medical system of any one of claims 1 to 24, wherein the plurality of inflatable portions includes at least three inflatable portions, and wherein, in the inflated operative state, a proximal-most inflatable portion and a distal-most inflatable portion have a greater external diameter than the rest of the plurality of inflatable portions disposed between the proximal most inflatable portion and the distal most inflatable portion.

26. The medical system of any one of claims 3 to 25, wherein each of the inflation orifices has a diameter or a longest dimension in the range of 0.5 mm to 10.0 mm, 0.5 mm to 5.0 mm, 0.5 mm to 2.0 mm, 0.5 mm to 1.0 mm, or 0.1 mm to 2.0 mm.
